SEQUENCE LISTING (6 sequences)

SEQ ID NO: 1 - DNA, 30 bp, Artificial Sequence
  Description of Artificial Sequence Synthetic oligonucleotide
cattacgagcgagatgtgtataagagacag

SEQ ID NO: 2 - DNA, 19 bp, Artificial Sequence
  Description of Artificial Sequence Synthetic oligonucleotide
ctgtctcttatacacatct

SEQ ID NO: 3 - DNA, 87 bp, Artificial Sequence
  Description of Artificial Sequence Synthetic oligonucleotide
acctgtgactgagacatctgaaggtgcaatcaggtgtcagtcttaaaggatcgataaggaagcggaagtagtggtctcgtcgtagtg

SEQ ID NO: 4 - DNA, 87 bp, Artificial Sequence
  Description of Artificial Sequence Synthetic oligonucleotide
cactacgacgagaccactacttccgcttccttatcgatcctttaagactgacacctgattgcaccttcagatgtctcagtcacaggt

SEQ ID NO: 5 - DNA, 20 bp, Artificial Sequence
  Description of Artificial Sequence Synthetic primer
acctgtgactgagacatctg

SEQ ID NO: 6 - DNA, 20 bp, Artificial Sequence
  Description of Artificial Sequence Synthetic primer
cactacgacgagaccactac

(19) United States
(12) Patent Application Publication
(10) Pub. No.: US 2020/0063213 A1
(43) Pub. Date: Feb. 27, 2020

(54) Methods of Amplifying DNA to Maintain Methylation Status

(71) Applicant: President and Fellows of Harvard College, Cambridge, MA (US)

(72) Inventors: Yunlong Cao, Cambridge, MA (US); Xiaoliang Sunney Xie, Lexington, MA (US)

(73) Assignee: President and Fellows of Harvard College, Cambridge, MA (US)

(21) Appl. No.: 16/488,658

(22) PCT Filed: Mar. 8, 2018
(86) PCT No.: PCT/US18/21453

§ 371 Date: Aug. 26, 2019

Related U.S. Application Data
(60) Provisional application No. 62/468,595, filed on Mar. 8, 2017.

(51) Int. Cl. C12Q 1/6886; C12P 19/34

(52) U.S. Cl. CPC C12Q 1/6886; C12P 19/34; C12Q 2600/112; C12Q 2600/154; C12Y 201/01037

(57) ABSTRACT

The present disclosure provides a method for making an amplified methylome by extending fragments and treating the extended fragments with a methyl transferase and source of methyl groups to transform hemi-methylated double stranded DNA to fully methylated double stranded DNA.

### RELATED APPLICATION DATA

This application claims priority to U.S. Provisional Application No. 62/468,595 filed on Mar. 8, 2017, which is hereby incorporated herein by reference in its entirety for all purposes

### STATEMENT OF GOVERNMENT INTERESTS

This invention was made with government support under 5DP1CA186693 from National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Mar. 30, 2018, is named 010498_01058_WO_SL.txt and is 1,858 bytes in size.

### BACKGROUND

### Field of the Invention

Embodiments of the present invention relate in general to methods and compositions for the amplification of DNA, such as DNA from a single cell, or cell free DNA, so as to maintain methylation information or status.

### Description of Related Art

Sodium bisulfite conversion of genomic DNA has been the gold standard for DNA methylation analysis. Treatment of DNA with bisulfite converts cytosine residues to uracil but leaves 5-methylcytosine residues unaffected. This method provides the ability to differentiate unmethylated versus methylated cytosines and provides a single nucleotide resolution map of DNA methylation status.

The major challenge in bisulfite conversion is the degradation and fragmentation of DNA that takes place concurrently with the conversion. The conditions necessary for complete conversions, such as long incubation times, elevated temperature, and high bisulfite concentration, can lead to the degradation and fragmentation of up to 90% of the incubated DNA. The degradation occurs as DNA depurination which results in random strand breaks. The extensive degradation is problematic and even more so such as when dealing with a limited amount of starting DNA or even single-cell level DNA. Low coverage single cell bisulfite sequencing has been achieved by directly performing bisulfite conversion on single cell followed by DNA amplification. Guo, H., et al. (2013). “Single-cell methylome landscapes of mouse embryonic stem cells and early embryos analyzed using reduced representation bisulfite sequencing.” Genome Res 23(12): 2126-2135; Smallwood, S. A., et al. (2014). “Single-cell genome-wide bisulfite sequencing for assessing epigenetic heterogeneity.” Nat Methods 11(8): 817-820.

The capability to perform high coverage genome methylation studies on single cell level DNA is important in studies where cell-to-cell variation and population heterogeneity play a key role, such as tumor growth, stem cell reprogramming, memory formation, embryonic development, etc. This is also important when the cell samples subject to analysis are precious or rare or in minute amounts, such as when the sample is a single cell or the genome, in whole or in part, of a single cell or cell free DNA.

Various known amplification methods, such as whole genome amplification methods result in amplified DNA where the methylation information or status from the original template is lost. Such whole genome amplification methods include multiple displacement amplification (MDA) which is a common method used in the art with genomic DNA from a single cell prior to sequencing and other analysis. In this method, random primer annealing is followed by extension taking advantage of a DNA polymerase with a strong strand displacement activity. The original genomic DNA from a single cell is amplified exponentially in a cascade-like manner to form hyperbranched DNA structures. Another method of amplifying genomic DNA from a single cell is described in Zong, C., Lu, S., Chapman, A. R., and Xie, X. S. (2012), Genome-wide detection of single-nucleotide and copy-number variations of a single human cell, Science 338, 1622-1626 which describes Multiple Annealing and Looping-Based Amplification Cycles (MALBAC). Another method known in the art is degenerate oligonucleotide primed PCR or DOP-PCR. Several other methods used with single cell genomic DNA include Cheung, V. G. and S. F. Nelson, Whole genome amplification using a degenerate oligonucleotide primer allows hundreds of genotypes to be performed on less than one nanogram of genomic DNA, Proceedings of the National Academy of Sciences of the United States of America, 1996. 93(25): p. 14676-9; Telenius, H., et al., Degenerate oligonucleotide-primed PCR: general amplification of target DNA by a single degenerate primer, Genomics, 1992. 13(3): p. 718-25; Zhang, L., et al., Whole genome amplification from a single cell: implications for genetic analysis. Proceedings of the National Academy of Sciences of the United States of America, 1992, 89(13): p. 5847-51; Lao, K., N. L. Xu, and N. A. Straus, Whole genome amplification using single-primer PCR, Biotechnology Journal, 2008, 3(3): p. 378-82; Dean, F. B., et al., Comprehensive human genome amplification using multiple displacement amplification, Proceedings of the National Academy of Sciences of the United States of America, 2002. 99(8): p. 5261-6; Lage, J. M., et al., Whole genome analysis of genetic alterations in small DNA samples using hyperbranched strand displacement amplification and array-CGH, Genome Research, 2003, 13(2): p. 294-307; Spits, C., et al., Optimization and evaluation of single-cell whole-genome multiple displacement amplification, Human Mutation, 2006, 27(5): p. 496-503; Gole, J., et al., Massively parallel polymerase cloning and genome sequencing of single cells using nanoliter microwells, Nature Biotechnology, 2013. 31(12): p. 1126-32; Jiang, Z., et al., Genome amplification of single sperm using multiple displacement amplification, Nucleic Acids Research, 2005, 33(10): p. e91; Wang, J., et al., Genome-wide Single-Cell Analysis of Recombination Activity and De Novo Mutation Rates in Human Sperm, Cell, 2012. 150(2): p. 402-12; Ni, X., Reproducible copy number variation patterns among single circulating tumor cells of lung cancer patients, PNAS, 2013, 110, 21082-21088; Navin, N., Tumor evolution inferred by single cell sequencing, Nature, 2011, 472 (7341):90-94; Evrony, G. D., et al., Single-neuron sequencing analysis of 11 retrotransposition and somatic mutation in the human brain, Cell, 2012. 151(3): p. 483-96; and McLean, J. S., et al., Genome of the pathogen Porphyromonas gingivalis recovered from a biofilm in a hospital sink using a high-throughput single-cell genomics platform, Genome Research, 2013. 23(5): p. 867-77. Methods directed to aspects of whole genome amplification are reported in WO 2012/166425, U.S. Pat. No. 7,718,403, US 2003/0108870 and U.S. Pat. No. 7,402,386.

However, a need exists for further methods of amplifying small amounts of genomic DNA, or DNA fragments, such as from a single cell or a small group of cells, or from cell free DNA, where the amplicons maintain the methylation information from the original template.

### SUMMARY

The present disclosure provides a method of producing or using DNA fragments which may then be subjected to denaturation and primer extension, such as single primer extension, for example using PCR conditions to produce two copies of hemi-methylated double stranded templates or fragments. The two copies of hemi-methylated double stranded templates or fragments are treated with a methyl transferase to methylate cytosine on the newly synthesized strand locations where the original template double stranded fragment was methylated, i.e. where methyl groups may have been removed as a result of the extension reaction. The process of primer extension to form hemi-methylated double stranded DNA and treating with a methyl transferase may be repeated to produce amplified double stranded DNA fragments having the methylation pattern of the original double stranded DNA template fragments. The population of amplified DNA fragments having the methylation characteristics of the original template fragments may be analyzed to determine the methylation characteristics.

Methods of fragmentation include those known in the art and include transposase fragmentation where a transposase or transposome is used to fragment the original or starting nucleic acid sequence, such as genomic DNA, fragments thereof, cell free DNA etc, and to attach a barcode sequence to each end of a cut or fragmentation site to facilitate the later computational rejoining of fragment sequences as part of a de novo assembly of the entire or whole methylome, if desired.

Further features and advantages of certain embodiments of the present disclosure will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 depicts in schematic a method of fragmenting a genomic nucleic acid sample followed by denaturation and primer extension and treatment with a methylation agent. The cycle is repeated 2 to 4 times to produce an amplified methylome which is then subjected to treatment with bisulfite or enzyme such as an ABOPEC family member or other reagent to alter cytocine to uracil.

FIG. 2 is a graph showing percentage of methylated cytosine in uniquely aligned reads of bisulfite sequencing results using 2×150 bp Miseq v2 kit (1,000,000 reads).

FIG. 3 is a graph showing performance of methyl-transferase DNMT1.

FIG. 4 is a schematic showing a cancer diagnosis method using amplification and methylation methods described herein.

FIG. 5 depicts data analyzing the performance on synthetic oligonucleotides of methylation sensitive restriction enzyme Clai in vitro to develop an optimal reaction buffer for the amplification and methylation reactions described herein.

FIG. 6 depicts data estimating the methyl-transfer efficiency of DNMT1 in certain buffer conditions.

FIG. 7 depicts data estimating the methyl-transfer efficiency of DNMT1 for a first and second round of denaturation and primer extension and treatment with a methylation agent in certain buffer conditions.

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The CpG sites or CG sites are regions of DNA where a cytosine nucleotide is followed by a guanine nucleotide in the linear sequence of bases along its 5′→3′ direction. In mammals, cytosines in CpG dinucleotides can be methylated to form 5-methylcytosine. Methylating the cytosine within a gene can change its expression, normally results in transcriptional silencing or suppression. In mammals, 70% to 80% of CpG cytosines are methylated and a total number of 28 million CpG sites exist in human. Mammalian DNA methylation of cytosines within the CpG dinucleotide context has been found to be associated with a number of key processes including embryogenesis, genomic imprinting, X-chromosome inactivation, aging, and carcinogenesis. In embryogenesis, DNA methylation patterns are largely erased and then re-established between generations in mammals. Almost all of the methylations from the parents are erased, first during gametogenesis, and again in early embryogenesis, with demethylation and remethylation occurring each time. In many disease processes, such as cancer, gene promoter CpG islands acquire abnormal hypermethylation, which results in transcriptional silencing that can be inherited by daughter cells following cell division.

The present disclosure is based on the recognition that an accurate genome methylation analysis is dependent on the maintenance of methylation information during the processing of the DNA, such as DNA in minute amounts or DNA from a single cell or cell free DNA. The present disclosure provides a method for amplifying DNA from a single cell or a small amount of DNA to produce amplicons having the methylation information or status of the original template DNA. According to one aspect, the methods described herein to enable the study of DNA methylation provide further methods of cancer diagnosis by comparing the methylation status of a DNA sample from an individual, such as a cell free DNA sample obtained from blood, with the methylation status of DNA indicating cancer, i.e. a standard. If the methylation status of the DNA sample correlates with the standard methylation status indicating cancer, then the individual is diagnosed with cancer. Methylation patterns for cancer DNA that may serve as a standard in the methods described herein are known to those of skill in the art as described in Vadakedath S, Kandi V (2016) DNA Methylation and Its Effect on Various Cancers: An Overview. J Mol Biomark Diagn S2:017. doi: 10.4172/2155-9929.S2-017 and A DataBase of Methylation Analysis on different type of cancers: MethHC: a database of DNA methylation and gene expression in human cancer. W. Y. Huang, S. D. Hsu, H. Y. Huang, Y. M. Sun, C. H. Chou, S. L. Weng, H. D. Huang* Nucleic Acids Res. 2015 January; 43(Database issue):D856-61 each of which is hereby incorporated by reference in its entirety.

According to one aspect, double stranded DNA fragments, such as cell free DNA, or DNA fragments produced from larger DNA, such as genomic DNA, are denatured into a first template single stranded DNA and a second template single stranded DNA followed by primer extension, such as single primer extension, of each of the first template single stranded DNA and the second template single stranded DNA to form a first hemi-methylated double stranded DNA and a second hemi-methylated double stranded DNA. The double stranded DNA is hemi-methylated insofar as the complementary strand created by primer extension lacks the methylation status of the original strand it has replaced. The hemi-methylated double stranded DNA is then treated with a methylation agent, such as a methyl transferase, such as DNMT1, to produce methylated double stranded DNA fragments which results in replication of the methylation status or information of the original template. If the methylation results in the original methylation status of the original template, the methylation of the hemi-methylated double stranded DNA is said to be fully methylated. This process of denaturing, primer extension to form hemi-methylated double stranded DNA and treatment with a methylation agent to restore methylation can be repeated a plurality of times, such as between 1 to 3 times, 1 to 4 times or 1 to 5 times i.e. the process can be carried out between 2 to 4 times, 2 to 5 times, or 2 to 6 times, to produced amplified fragments having the methylation status or information of the original template fragments. The amplified fragments having the methylation status or information of the original template fragments may then be treated with a reagent that converts cytosine to uracil as is known in the art and the treated amplified fragments may then be sequenced as is known in the art, for example using high throughput sequencing methods, to determine the nature and extent of methylation, methylation patterns, presence or absence of methylation, etc. According to one aspect, the amplification process produces sufficient amount of amplicons with the original methylation status of the original template so as to offset the loss of DNA due to degradation by treatment with a reagent that converts cytosine to uracil, such as by degradation by bisulfite treatment, or allele drop out when performing PCR reactions, while still having sufficient DNA for methylation analysis.

Methylating agents are known to those of skill in the art and will become apparent based on the present disclosure. Methylating agents may be a methyl-transferase. One exemplary methylating agent is DNMT1. DNMT1 is the most abundant DNA methyl-transferase in mammalian cells and is considered to be the key maintenance methyltransferase due to its ability to predominantly methylate hemimethylated CpG di-nucleotides in the mammalian genome. This enzyme is 7 to 100-fold more active on hemimethylated DNA as compared with the unmethylated substrate in vitro. By combining a single round of PCR reaction and DNMT1 incubation on genomic DNA, one can achieve the replication of genomic DNA methylation status. Furthermore, the methylation replication loops can be performed multiple times which results in up to 32-fold increase of starting DNA for bisulfite conversion or enzyme conversion such as APOBEC or other agent that converts cytosine to uracil. Additional useful methylating agents include DNMT3a and DNMT3b which are mammalian methyl transferases. Additional useful methylating agents include DRM2, MET1, and CMT3 which are plant methyl transferases. Additional useful methylating agents include Dam which is a bacterial methyl transferase. According to one aspect, it is to be understood that DNMT1 or other suitable methyltransferases are used with a source of methyl and may be used with or without cofactors known to those of skill in the art. DNMT1 works in vitro at 95% efficiency without a cofactor, however, DNMT1 may be used with a cofactor such as NP95(Uhrf1) as described in Bashtrykov P1, Jankevicius G, Jurkowska R Z, Ragozin S, Jeltsch A. The UHRF1 protein stimulates the activity and specificity of the maintenance DNA methyltransferase DNMT1 by an allosteric mechanism. J Biol Chem. 2014 hereby incorporated by reference in its entirety.

According to one aspect, a methyl-transferase, such DNMT1, may require conditions, such as buffer conditions, that do not include ions (such as cations), such as magnesium ions or manganese ions which may be a component or condition of a PCR reaction used for primer extension. One of skill will readily understand the nature and extent of ions, such as cations, that are used for primer extension or PCR reactions. According to one aspect, a chelating agent, such as EDTA, is used after the primer extension step to chelate ions, such as magnesium ions in order for the methylation step to be carried out. One of skill will understand that the chelating step is intended to chelate ions that are used in the primer extension step but that may inhibit the methylation step. It is to be understood that under some conditions, magnesium ions may be present in the reaction media during the methylation step. However, in a certain embodiment such as where the primer extension step and methylation step is carrier out in the same vessel with the same media, a purification step is not needed as a chelating agent such as EDTA is used to chelate magnesium in an equal molar fashion to provide a magnesium free buffer for the methyl-transfer reaction. Magnesium is replenished back to the reaction for the next primer extension step under PCR conditions after the completion of methyl-transfer reactions. Exemplary chelating agents include iminodisuccinic acid (IDS), polyaspartic acid, ethylenediamine-N, N′-disuccinic acid (EDDS), Methylglycinediacetic acid, aminopolycarboxylate-based chelates, tetrasodium salt or N-diacetic acid.

Reagents to convert cytosine to uracil are known to those of skill in the art and include bisulfite reagents such as sodium bisulfite, potassium bisulfite, ammonium bisulfite, magnesium bisulfite, sodium metabisulfite, potassium metabisulfite, ammonium metabisulfite, magnesium metabisulfite and the like. Enzymatic reagents to convert cytosine to uracil, i.e. cytosine deaminases, include those of the ABOPEC family, such as APOBEC-seq or APOBEC3A. The APOBEC family members are cytidine deaminases that convert cytosine to uracil while maintaining 5-methyl cytosine, i.e. without altering 5-methyl cytosine. Such enzymes are described in US 2013/0244237 and may be available from New England Biolabs. Other enzymatic reagents will become apparent to those of skill in the art based on the present disclosure.

A DNA sample treated with a bisulfite reagent, such as sodium bisulfite, can convert cytosine to uracil and leave the 5-methylcytosine (mC) unchanged. Thus after bisulfite treatment, 5-mC in the DNA remains as cytosine and unmodified cytosine will be changed to uracil. The bisulfite treatment can be performed by commercial kits such as the Imprint DNA Modification Kit (Sigma), EZ DNA Methylation-Direct™ Kit (ZYMO) etc. Once DNA bisulfite conversion is complete, single stranded DNA is captured, desulphonated and cleaned. The bisulfite-treated DNA can be captured by purification columns or magnetic beads. The bisulfite-treated DNA is further desulphonated with an alkalized solution, preferably sodium hydroxide. The DNA is then eluted and collected into a PCR tube. Bisulfite-treated single stranded DNA can be converted into dsDNA through an enzyme-catalyzed DNA strand synthesis with appropriate primers. Suitable enzymes include Bst DNA polymerase, exonuclease deficient Klenow DNA polymerase large fragment, phi29 DNA polymerase, T4 DNA polymerase, T7 DNA polymerase, HIV-1 reverse transcriptase, M-MLV reverse transcriptase, AMV reverse transcriptase and the like. These enzymes will recognize the uracil in the ssDNA template as thymine and add an adenine to the complimentary strand. Further polymerase extension on the complimentary strand will result in replication of the original bisulfite treated ssDNA template except substituting uracil with thymine. Thus, by identifying all Cytosine to Thymine conversion and Guanine to Adenine conversion (complementary strand) through comparing to the reference genome, all unmodified cytosine can be identified while the remaining cytosine are considered to be methylated.

For single-cell whole methylome analysis, random primers are used, preferably 6-8 mers and most preferably hexamers. For cancer diagnosis, a set (20+) of selected bisulfite PCR primers (designed to amplify bisulfite treated DNA) which targets different cancer differential methylated genes (genes that are only methylated or unmethylated in certain kind of cancer) are used. Exemplary cancer-related genes include SEPT9 gene, TMEM106A, NCS1, UXS1, HORMAD2, REC8, DOCK8, CDKL5, and the like. Further cancer related genes will become apparent to those of skill in the art based upon the present disclosure. The selection of primers are based on standard cancer methylation data. Different combinations of the methylation status of the targeted genes identifies a particular cancer type present within an individual.

Accordingly, methods described herein can be practiced on a nucleic acid sample, such as a small amount of genomic DNA or a limited amount of DNA such as cell free DNA, such as a genomic sequence or genomic sequences obtained from a single cell or a plurality of cells of the same cell type or from an embryo, a tissue, fluid or blood sample obtained from an individual or a substrate. According to certain aspects of the present disclosure, the nucleic acid sample can be within an unpurified or unprocessed lysate from a single cell. According to certain aspects of the present disclosure, the nucleic acid sample can be cell-free DNA such as is present within a fluid biological sample such as blood. Nucleic acids to be subjected to the methods disclosed herein need not be purified, such as by column purification, prior to being contacted with the various reagents and under the various conditions as described herein.

According to certain aspect, the method described herein may be referred to as a methylation amplification method or methylome replication loops with methyl-transferase (MERLOT). Methods described herein provide pre-amplification of single-cell level genomic DNA or small amounts of DNA while maintaining methylation information or status of the original template dsDNA sequence. According to one exemplary aspect, the method combines a single round of PCR reaction and human methyl-transferase DNMT1 incubation on DNA to achieve the replication of DNA methylation status. According to one aspect between a 2 fold and 32 fold increase, a 2 fold and 19 fold increase, a 2 fold and 18 fold increase, a 2 fold and 17 fold increase, a 2 fold and 16 fold increase, a 2 fold and 8 fold increase, a 2 fold and 4 fold increase in the amount of starting DNA for bisulfite conversion is achieved by performing the methylome replication loop for multiple times. Such amplified DNA can compensate for the loss of DNA during bisulfite conversion or whole genome amplification and results in greater efficiency in characterizing methylation status of DNA, such as single-cell level DNA or small amounts of DNA.

Embodiments of the present invention utilize methods for making DNA fragments, for example, DNA fragments from the whole genome of a single cell or a small amount of DNA or DNA from an embryo which may then be subjected to the amplification method described herein to maintain methylation information followed by sequencing methods known to those of skill in the art and as described herein.

Methods of making DNA fragments from an original DNA sample are known to those of skill in the art. One approach includes sonication followed by end repair and adapter sequence ligation. For cancer diagnosis, a set (20+) of selected targeted PCR primers (for normal DNA) are used to create DNA amplicons with priming sites on both ends. The gene targets include SEPT9 gene, TMEM106A, NCS1, UXS1, HORMAD2, REC8, DOCK8, CDKL5, and the like.

According to one exemplary aspect, methods are described of making nucleic acid fragments using an enzyme such as Tn5. Such methods are known in the art and include those practiced using the illumina Nextera kit. According to one exemplary aspect, methods described herein utilize a transposome library and a method referred to as “tagmentation” to the extent that fragments are created from a larger dsDNA sequence where the fragments are tagged with primers to be used in single primer extension and amplification. In general, a transposase as part of a transposome is used to create a set of double stranded genomic DNA fragments. According to certain aspects, the transposases have the capability to bind to transposon DNA and dimerize when contacted together, such as when being placed within a reaction vessel or reaction volume, forming a transposase/transposon DNA complex dimer called a transposome. Each transposon DNA of the transposome includes a double stranded transposase binding site and a first nucleic acid sequence including an amplification promoting sequence, such as a specific priming site (“primer binding site”) or a transcription promoter site. The first nucleic acid sequence may be in the form of a single stranded extension.

The transposomes have the capability to randomly bind to target locations along double stranded nucleic acids, such as double stranded genomic DNA, forming a complex including the transposome and the double stranded genomic DNA. The transposases in the transposome cleave the double stranded genomic DNA, with one transposase cleaving the upper strand and one transposase cleaving the lower strand. Each of the transposon DNA in the transposome is attached to the double stranded genomic DNA at each end of the cut site, i.e. one transposon DNA of the transposome is attached to the left hand cut site and the other transposon DNA of the transposome is attached to the right hand cut site. In this manner, the left hand cut site and the right hand cut site are provided with a primer binding site.

According to certain aspects, a plurality of transposase/transposon DNA complex dimers, i.e. transposomes, bind to a corresponding plurality of target locations along a double stranded genomic DNA, for example, and then cleave the double stranded genomic DNA into a plurality of double stranded fragments with each fragment having transposon DNA with a primer binding site attached at each end of the double stranded fragment. In this manner, the primer binding sites may be used in a single primer extension reaction.

According to one aspect, the transposon DNA is attached to the double stranded genomic DNA and a single stranded gap exists between one strand of the genomic DNA and one strand of the transposon DNA. According to one aspect, gap extension is carried out to fill the gap and create a double stranded connection between the double stranded genomic DNA and the double stranded transposon DNA. According to one aspect, a nucleic acid sequence including the transposase binding site and the amplification promoting sequence of the transposon DNA is attached at each end of the double stranded fragment. According to certain aspects, the transposase is attached to the transposon DNA which is attached at each end of the double stranded fragment. According to one aspect, the transposases are removed from the transposon DNA which is attached at each end of the double stranded genomic DNA fragments.

According to one aspect of the present disclosure, the double stranded genomic DNA fragments produced by the transposases which have the transposon DNA attached at each end of the double stranded genomic DNA fragments are then gap filled and extended by way of the primer binding site using the transposon DNA as a template. Accordingly, a double stranded nucleic acid extension product is produced which includes the double stranded genomic DNA fragment and a double stranded transposon DNA including an amplification promoting sequence, i.e, primer extension sequence, at each end of the double stranded genomic DNA.

At this stage, a double stranded nucleic acid extension product including the genomic DNA fragment and the amplification promoting sequence can be subjected to primer extension using methods known to those of skill in the art to produce a pair of hemi-methylated double stranded DNA. The pair of hemi-methylated double stranded DNA is then incubated with a methylation agent, such as a methyl transferase, such as DNMT1, and a source of methyl groups to place methyl groups on the strand that was created by primer extension so as to match the methylation of the original template strand. In this manner, the method can be said to have added methyl groups or restored methyl groups or restored the methylation information or status of the original template strand that was lost due to the single primer extension reaction to create the complementary strand.

The primer extension includes the use of single or multiple primer extension. The single primer extension includes the use of a promoting sequence that can be a specific primer binding site at each end of the double stranded genomic DNA. The reference to a “specific” primer binding site indicates that the two primer binding sites have the same sequence and so a primer of a common sequence can be used for extension of all fragments. PCR primer sequences and reagents can be used for extension. The extension method can be carried out any number of times as desired and as to maximize the creation of amplicons having the methylation information of the original template fragment.

The amplicons can then be collected and/or purified prior to further analysis. The amplicons can be amplified and/or sequenced using methods known to those of skill in the art. Once sequenced, the methylation information of the fragments can be analyzed using methods known to those of skill in the art, and can then be compared with methylation standards corresponding to certain diseases, for example, as a method of diagnosing an individual with a certain disease.

Embodiments of the present disclosure are directed to a method of producing DNA amplicons having the methylation status or information of the original DNA template, which can be lost due to amplification and/or primer extension reactions to create a complementary strand. The DNA may be a small amount of genomic DNA or a limited amount of DNA such as a genomic sequence or genomic sequences obtained from a single cell or a plurality of cells of the same cell type or from a tissue, fluid or blood sample, i.e. circulating DNA, obtained from an individual or a substrate. According to certain aspects of the present disclosure, the methods described herein utilize tagmentation methods of fragmenting DNA using a transposase including an extension primer to produce dsDNA which includes extension primer sites, or use targeted PCR to produce amplicons of targeted genes. These fragments can be denatured into individual strands and extended and the methylation information restored. This process can be repeated many times to produce an amplified methylome which can be subjected to bisulfite conversion or ABOPEC treatment. The bisulfite converted amplified methylome can then be subjected to amplification and/or sequencing, for example, using high throughput sequencing platforms known to those of skill in the art. The methylation status can be analyzed according to methods known in the art, such as by analyzing the sequencing information.

Methods described herein have particular application in biological systems or tissue samples characterized by highly heterogeneous cell populations such as tumor and neural masses. The methods described herein can utilize varied sources of DNA materials, including genetically heterogeneous tissues (e.g. cancers), rare and precious samples (e.g. embryonic stem cells), and non-dividing cells (e.g. neurons) and the like, as well as, sequencing platforms and genotyping methods known to those of skill in the art.

According to one aspect, DNA, such as genomic nucleic acid obtained from a lysed single cell, is obtained. A plurality or library of transposomes is used to cut the DNA into double stranded fragments. Each transposome of the plurality or library is a dimer of a transposase bound to a transposon DNA, i.e. each transposome includes two separate transposon DNA. Each transposon DNA of a transposome includes a transposase binding site and an amplification or extension facilitating sequence, such as a specific primer binding site, for example for single primer extension methods.

The transposon DNA becomes attached to the upper and lower strands of each double stranded fragment at each cut or fragmentation site. The double stranded fragments are then processed to fill gaps. The fragments are then subject to single primer extension to produce hemi-methylated dsDNA which is then subjected to a methyl transferase to add a methyl group at various locations to replicate the methylation status of the original dsDNA template. This process is repeated to produce a population of amplified template fragments having the methylation characteristics of the original template fragments. Methylation characteristics may be determined. According to one aspect, the fragments may be amplified and/or sequenced and methylation characteristics may be determined.

In certain aspects, primer extension amplification is achieved using PCR conditions. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). The term “polymerase chain reaction” (“PCR”) of Mullis (U.S. Pat. Nos. 4,683,195, 4,683,202, and 4,965,188) refers to a method for increasing the concentration of a segment of a target sequence without cloning or purification. This process for amplifying the target sequence includes providing oligonucleotide primers with the desired target sequence and amplification reagents, followed by a precise sequence of thermal cycling in the presence of a polymerase (e.g., DNA polymerase). The primers are complementary to their respective strands (“primer binding sequences”) of the double stranded target sequence. To effect amplification, the double stranded target sequence is denatured and the primers then annealed to their complementary sequences within the target molecule. Following annealing, the primers are extended with a polymerase so as to form a new pair of complementary strands. If desired, the steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. According to the present disclosure, after one cycle, the resulting amplicons are treated with a methyl adding reagent or enzyme, such a methyl transferase, such as DNMT1 to add methyl groups to the double stranded nucleic acid fragments. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”) and the target sequence is said to be “PCR amplified.” The PCR amplification reaches saturation when the double stranded DNA amplification product accumulates to a certain amount that the activity of DNA polymerase is inhibited. Once saturated, the PCR amplification reaches a plateau where the amplification product does not increase with more PCR cycles.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. Methods and kits for performing PCR are well known in the art. All processes of producing replicate copies of a polynucleotide, such as PCR or gene cloning, are collectively referred to herein as replication. A primer can also be used as a probe in hybridization reactions, such as Southern or Northern blot analyses.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reactions are commercially available. Primers useful to amplify sequences from a particular gene region are preferably complementary to, and hybridize specifically to sequences in the target region or in its flanking regions and can be prepared using methods known to those of skill in the art. Nucleic acid sequences generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The terms “PCR product,” “PCR fragment,” and “amplification product” refer to the resultant mixture of compounds after one or more cycles of the PCR steps of denaturation, annealing and extension are complete. These terms encompass the case where there has been amplification of one or more segments of one or more target sequences.

The term “amplification reagents” may refer to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

According to one aspect, a method of making an amplified methylome for bisulfite treatment or APOBEC treatment is provided which includes contacting double stranded genomic DNA from a single cell with Tn5 transposases each bound to a transposon DNA, wherein the transposon DNA includes a double-stranded 19 bp transposase (Tnp) binding site and a first nucleic acid sequence including a primer binding site to form a transposase/transposon DNA complex dimer called a transposome. The first nucleic acid sequence may be in the form of a single stranded extension. According to one aspect, the first nucleic acid sequence may be an overhang, such as a 5′ overhang, wherein the overhang includes a priming site. The overhang can be of any length suitable to include a priming site as desired. The transposome bind to target locations along the double stranded genomic DNA and cleave the double stranded genomic DNA into a plurality of double stranded fragments, with each double stranded fragment having a first complex attached to an upper strand by the Tnp binding site and a second complex attached to a lower strand by the Tnp binding site. The transposon binding site, and therefore the transposon DNA, is attached to each 5′ end of the double stranded fragment. According to one aspect, the Tn5 transposases are removed from the complex. The double stranded fragments are extended along the transposon DNA to make a double stranded extension product having specific primer binding sites at each end of the double stranded extension product. According to one aspect, a gap which may result from attachment of the Tn5 transposase binding site to the double stranded genomic DNA fragment may be filled. The gap filled double stranded extension product is denatured into single strands, each of which are subject to single primer extension to produce hemi-methylated dsDNA followed by treatment with a methyl transferase such as DNMT1 so as to add methyl groups to the hemi-methylated dsDNA. The denaturing, primer extension and methylation steps are repeated a plurality of times to create amplicons of the original template dsDNA with the original methylation status. The amplicons may then be treated with bisulfite or APOBEC or other reagent that converts cytosine to uracil but without altering 5-methyl cytosine. The treated amplicon DNA may then be subjected to multiple rounds of random priming amplification, such as by using Klenow fragement exo- or Bst large fragments followed by about 13 to 18 rounds of PCR reaction with adapters, such as with illumina adapters. A suitable amplification protocol is described in Stephen J Clark, Sébastien A Smallwood, Heather J Lee, Felix Krueger, Wolf Reik & Gavin Kelsey. Genome-wide base-resolution mapping of DNA methylation in single cells using single-cell bisulfite sequencing (scBS-seq). Nature Protocols (2017) hereby incorporated by reference in its entirety. Pico Methyl-Seq™ Library Prep Kit from ZYMO may also be used.

According to certain aspects, an exemplary transposon system includes Tn5 transposase, Mu transposase, Tn7 transposase or IS5 transposase and the like. Other useful transposon systems are known to those of skill in the art and include Tn3 transposon system (see Maekawa, T., Yanagihara, K., and Ohtsubo, E. (1996), A cell-free system of Tn3 transposition and transposition immunity, Genes Cells 1, 1007-1016), Tn7 transposon system (see Craig, N. L. (1991), Tn7: a target site-specific transposon, Mol. Microbiol. 5, 2569-2573), Tn10 tranposon system (see Chalmers, R., Sewitz, S., Lipkow, K., and Crellin, P. (2000), Complete nucleotide sequence of Tn10, J. Bacteriol 182, 2970-2972), Piggybac transposon system (see Li, X., Burnight, E. R., Cooney, A. L., Malani, N., Brady, T., Sander, J. D., Staber, J., Wheelan, S. J., Joung, J. K., McCray, P. B., Jr., et al. (2013), PiggyBac transposase tools for genome engineering, Proc. Natl. Acad. Sci. USA 110, E2279-2287), Sleeping beauty transposon system (see Ivics, Z., Hackett, P. B., Plasterk, R. H., and Izsvak, Z. (1997), Molecular reconstruction of Sleeping Beauty, a Tcl-like transposon from fish, and its transposition in human cells, Cell 91, 501-510), Tol2 transposon system (see Kawakami, K. (2007), Tol2: a versatile gene transfer vector in vertebrates, Genome Biol. 8 Suppl. 1, S7.)

DNA may be obtained from a biological sample. As used herein, the term “biological sample” is intended to include, but is not limited to, tissues, cells, biological fluids and isolates thereof, isolated from a subject, as well as tissues, cells and fluids present within a subject.

DNA may be obtained from a single cell or a small population of cells. The DNA may be from any species or organism including but not limited to human, animal, plant, yeast, viral, eukaryotic and prokaryotic DNA. In a particular aspect, embodiments are directed to methods for the amplification of substantially the entire genome without loss of representation of specific sites and resulting in an amplified methylome (herein defined as “whole genome amplification”). In a specific embodiment, whole genome amplification comprises amplification of substantially all fragments or all fragments of a genomic library. In a further specific embodiment, “substantially entire” or “substantially all” refers to about 80%, about 85%, about 90%, about 95%, about 97%, or about 99% of all sequences in a genome.

According to one aspect, the DNA sample is genomic DNA, micro dissected chromosome DNA, yeast artificial chromosome (YAC) DNA, plasmid DNA, cosmid DNA, phage DNA, P1 derived artificial chromosome (PAC) DNA, or bacterial artificial chromosome (BAC) DNA, mitochondrial DNA, chloroplast DNA, forensic sample DNA, or other DNA from natural or artificial sources to be tested. In another preferred embodiment, the DNA sample is mammalian DNA, plant DNA, yeast DNA, viral DNA, or prokaryotic DNA. In yet another preferred embodiment, the DNA sample is obtained from a human, bovine, porcine, ovine, equine, rodent, avian, fish, shrimp, plant, yeast, virus, or bacteria. Preferably the DNA sample is genomic DNA.

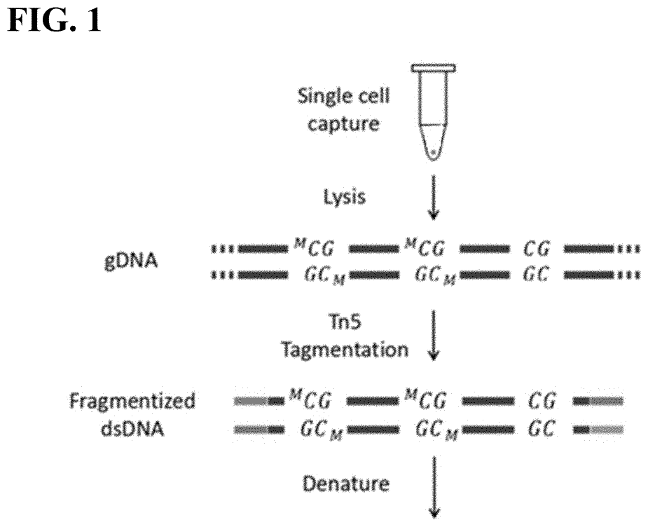

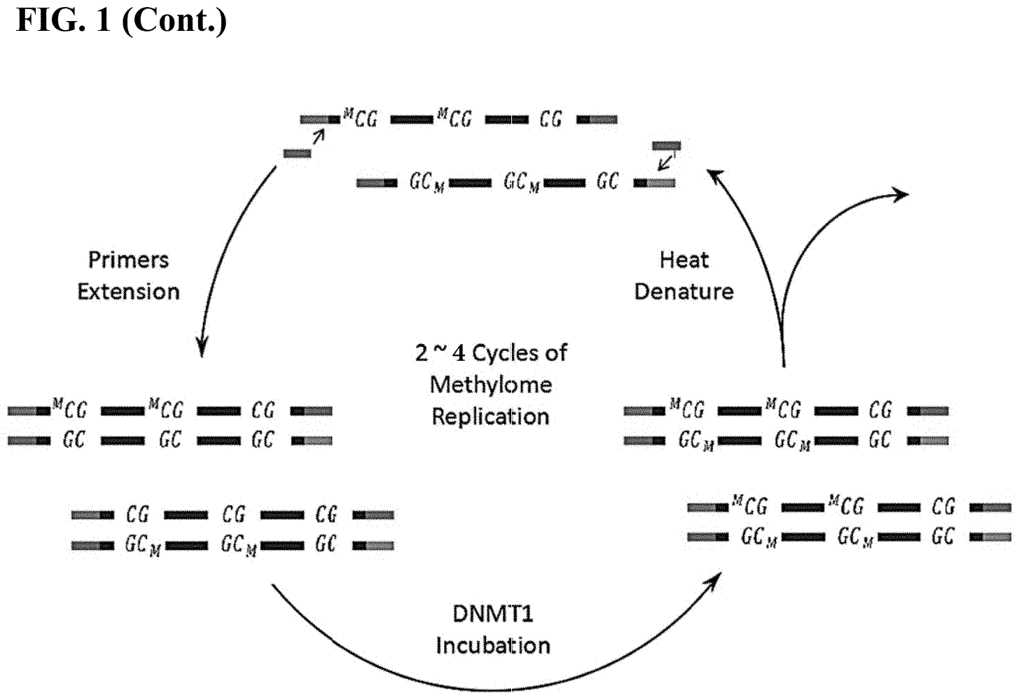

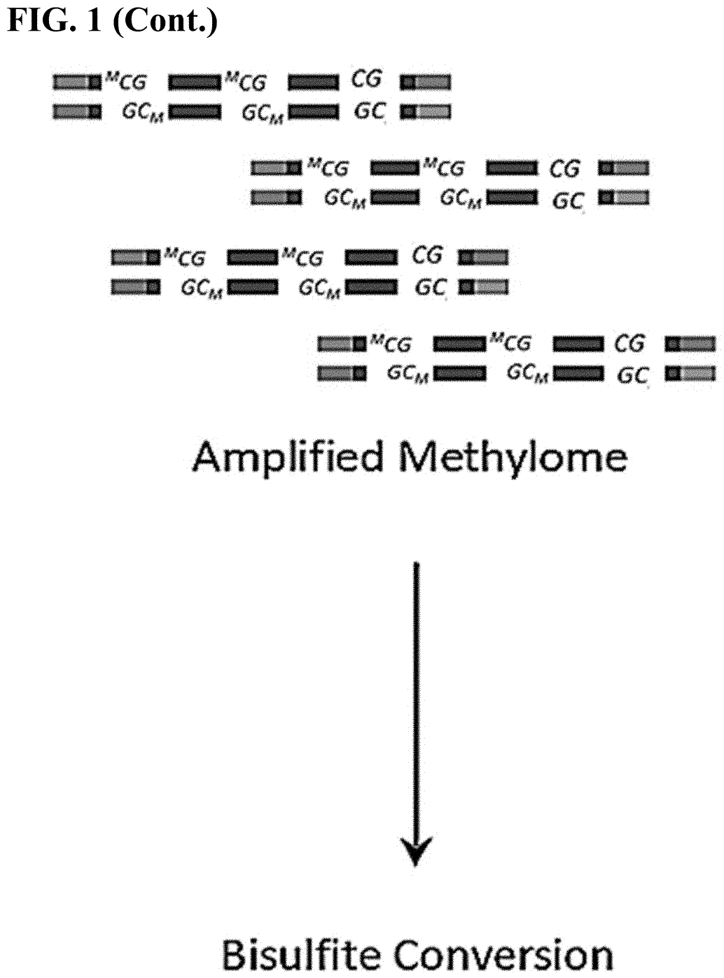

According to certain exemplary aspects, a transposition system is used to make nucleic acid fragments for multiple primer extension and methylation reactions to produce an amplified methylome for bisulfite treatment, for example in a single reaction vessel. According to an exemplary embodiment shown in FIG. 1, a single cell is first captured into lysis buffer in a PCR tube to release gDNA. The genomic DNA is than subjected to Tn5 tagmentation and fragmented into about 1 kb dsDNA with complementary PCR priming sites on both ends. The resulted dsDNA fragments are heat denatured into ssDNA followed by primer extension to form hemi-methylated dsDNA. The hemi-methylated dsDNA are incubated with DNMT1 and SAM for 3 hours to become fully methylated dsDNA, which results in a replication of the methylation status of the original template. The fully methylated dsDNA are heat denatured again and the replication loop is repeated. The replication loop may be repeated for 1 to 20 times, 1 to 10 times, or 1 to 5 times. The amplified product is treated with sodium bisulfite and is ready for downstream analysis.

Particular Tn5 transposition systems are described and are available to those of skill in the art. See Goryshin, I. Y. and W. S. Reznikoff, Tn5 in vitro transposition. The Journal of biological chemistry, 1998. 273(13): p. 7367-74; Davies, D. R., et al., Three-dimensional structure of the Tn5 synaptic complex transposition intermediate. Science, 2000. 289(5476): p. 77-85; Goryshin, I. Y., et al., Insertional transposon mutagenesis by electroporation of released Tn5 transposition complexes. Nature biotechnology, 2000. 18(1): p. 97-100 and Steiniger-White, M., I. Rayment, and W. S. Reznikoff, Structure/function insights into Tn5 transposition. Current opinion in structural biology, 2004. 14(1): p. 50-7 each of which are hereby incorporated by reference in their entireties for all purposes. Kits utilizing a Tn5 transposition system for DNA library preparation and other uses are known. See Adey, A., et al., Rapid, low-input, low-bias construction of shotgun fragment libraries by high-density in vitro transposition. Genome biology, 2010. 11(12): p. R119; Marine, R., et al., Evaluation of a transposase protocol for rapid generation of shotgun high-throughput sequencing libraries from nanogram quantities of DNA. Applied and environmental microbiology, 2011. 77(22): p. 8071-9; Parkinson, N. J., et al., Preparation of high-quality next-generation sequencing libraries from picogram quantities of target DNA. Genome research, 2012. 22(1): p. 125-33; Adey, A. and J. Shendure, Ultra-low-input, tagmentation-based whole-genome bisulfite sequencing. Genome research, 2012. 22(6): p. 1139-43; Picelli, S., et al., Full-length RNA-seq from single cells using Smart-seq2. Nature protocols, 2014. 9(1): p. 171-81 and Buenrostro, J. D., et al., Transposition of native chromatin for fast and sensitive epigenomic profiling of open chromatin, DNA-binding proteins and nucleosome position. Nature methods, 2013, each of which is hereby incorporated by reference in its entirety for all purposes. See also WO 98/10077, EP 2527438 and EP 2376517 each of which is hereby incorporated by reference in its entirety. A commercially available transposition kit is marketed under the name NEXTERA and is available from Illumina.

The term “genome” as used herein is defined as the collective gene set carried by an individual, cell, or organelle. The term “genomic DNA” as used herein is defined as DNA material comprising the partial or full collective gene set carried by an individual, cell, or organelle. Aspects of the present disclosure include the use of cell free DNA.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. The term also refers to both double- and single-stranded molecules. Unless otherwise specified or required, any embodiment of this invention that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule. This alphabetical representation can be input into databases in a computer having a central processing unit and used for bioinformatics applications such as functional genomics and homology searching.

The terms “DNA,” “DNA molecule” and “deoxyribonucleic acid molecule” refer to a polymer of deoxyribonucleotides. DNA can be synthesized naturally (e.g., by DNA replication). RNA can be post-transcriptionally modified. DNA can also be chemically synthesized. DNA can be single-stranded (i.e., ssDNA) or multi-stranded (e.g., double stranded, i.e., dsDNA).

The terms “nucleotide analog,” “altered nucleotide” and “modified nucleotide” refer to a non-standard nucleotide, including non-naturally occurring ribonucleotides or deoxyribonucleotides. In certain exemplary embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the terms “complementary” and “complementarity” are used in reference to nucleotide sequences related by the base-pairing rules. For example, the sequence 5′-AGT-3′ is complementary to the sequence 5′-ACT-3′. Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. The degree of complementarity between nucleic acid strands has significant effects on the efficiency and strength of hybridization between nucleic acid strands.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tₘ” refers to the melting temperature of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tₘ=81.5+0.41 (% G+C), when a nucleic acid is in aqueous solution at 1 M NaCl (See, e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted.

“Low stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 mg/ml denatured salmon sperm DNA followed by washing in a solution comprising 5×SSPE, 0.1% SDS at 42° C. when a probe of about 500 nucleotides in length is employed.

“Medium stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5×Denhardt's reagent and 100 mg/ml denatured salmon sperm DNA followed by washing in a solution comprising 1.Ox SSPE, 1.0% SDS at 42° C. when a probe of about 500 nucleotides in length is employed.

“High stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA, pH adjusted to 7.4 with NaOH), 0.5% SDS, 5×Denhardt's reagent and 100 mg/ml denatured salmon sperm DNA followed by washing in a solution comprising 0.1×SSPE, 1.0% SDS at 42° C. when a probe of about 500 nucleotides in length is employed.

In certain exemplary embodiments, cells are identified and then a single cell or a plurality of cells is isolated. Cells within the scope of the present disclosure include any type of cell where understanding the DNA content is considered by those of skill in the art to be useful. A cell according to the present disclosure includes a cancer cell of any type, hepatocyte, oocyte, embryo, stem cell, iPS cell, ES cell, neuron, erythrocyte, melanocyte, astrocyte, germ cell, oligodendrocyte, kidney cell and the like. According to one aspect, the methods of the present invention are practiced with the cellular DNA from a single cell. A plurality of cells includes from about 2 to about 1,000,000 cells, about 2 to about 10 cells, about 2 to about 100 cells, about 2 to about 1,000 cells, about 2 to about 10,000 cells, about 2 to about 100,000 cells, about 2 to about 10 cells or about 2 to about 5 cells.

Nucleic acids processed by methods described herein may be DNA and they may be obtained from any useful source, such as, for example, a human sample. In specific embodiments, a double stranded DNA molecule is further defined as comprising a genome, such as, for example, one obtained from a sample from a human. The sample may be any sample from a human, such as blood, serum, plasma, cerebrospinal fluid, cheek scrapings, nipple aspirate, biopsy, semen (which may be referred to as ejaculate), urine, feces, hair follicle, saliva, sweat, immunoprecipitated or physically isolated chromatin, and so forth. In specific embodiments, the sample comprises a single cell. In specific embodiments, the sample includes only a single cell.

A nucleic acid used in the invention can also include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

In particular embodiments, the amplified metylome which has been treated with bisulfite or APOBEC or other reagent that converts cytosine to uracil and analyzed for methylation provides diagnostic or prognostic information. For example, the amplified metylome which has been treated with bisulfite or APOBEC or other reagent that converts cytosine to uracil and analyzed for methylation may provide genomic copy number and/or sequence information, genomic imprinting information, allelic variation information, cancer diagnosis, prenatal diagnosis, paternity information, disease diagnosis, detection, monitoring, and/or treatment information, sequence information, and so forth.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, such that each single cell is placed in a single well.

Methods for manipulating single cells are known in the art and include fluorescence activated cell sorting (FACS), flow cytometry (Herzenberg., PNAS USA 76:1453-55 1979), micromanipulation and the use of semi-automated cell pickers (e.g. the Quixell™ cell transfer system from Stoelting Co.). Individual cells can, for example, be individually selected based on features detectable by microscopic observation, such as location, morphology, or reporter gene expression. Additionally, a combination of gradient centrifugation and flow cytometry can also be used to increase isolation or sorting efficiency.

Once a desired cell has been identified, the cell is lysed to release cellular contents including DNA, using methods known to those of skill in the art. The cellular contents are contained within a vessel or a collection volume. In some aspects of the invention, cellular contents, such as genomic DNA, can be released from the cells by lysing the cells. Lysis can be achieved by, for example, heating the cells, or by the use of detergents or other chemical methods, or by a combination of these. However, any suitable lysis method known in the art can be used. For example, heating the cells at 72° C. for 2 minutes in the presence of Tween-20 is sufficient to lyse the cells. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313). Amplification of genomic DNA according to methods described herein can be performed directly on cell lysates, such that a reaction mix can be added to the cell lysates. Alternatively, the cell lysate can be separated into two or more volumes such as into two or more containers, tubes or regions using methods known to those of skill in the art with a portion of the cell lysate contained in each volume container, tube or region. Genomic DNA contained in each container, tube or region may then be amplified by methods described herein or methods known to those of skill in the art.

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

Primers include those that are specific to selected target loci, such DNA associated with a disease, such as cancer, and may be referred to as target loci specific primers, disease specific primers or cancer specific primers. Using such target loci specific primers or disease specific primers or cancer specific primers allows the amplification of target loci such as disease specific DNA or cancer specific DNA thereby allowing identification of disease specific DNA or cancer specific DNA so as to diagnose an individual with the disease or cancer.

The expression “amplification” or “amplifying” refers to a process by which extra or multiple copies of a particular polynucleotide are formed.

The amplified methylome that has been treated with bisulfite or APOBEC or other reagent that converts cytosine to uracil may be amplified, sequenced and analyzed using methods known to those of skill in the art. Determination of the sequence of a nucleic acid sequence of interest can be performed using a variety of sequencing methods known in the art including, but not limited to, sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

Further sequencing methods include high-throughput screening methods, such as Applied Biosystems' SOLiD sequencing technology, or Illumina's Genome Analyzer. In one aspect of the invention, the DNA can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

“Shotgun sequencing” refers to a method used to sequence very large amount of DNA (such as the entire genome). In this method, the DNA to be sequenced is first shredded into smaller fragments which can be sequenced individually. The sequences of these fragments are then reassembled into their original order based on their overlapping sequences, thus yielding a complete sequence. “Shredding” of the DNA can be done using a number of difference techniques including restriction enzyme digestion or mechanical shearing. Overlapping sequences are typically aligned by a computer suitably programmed. Methods and programs for shotgun sequencing a cDNA library are well known in the art.

The methods described herein are useful in the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the genomic DNA in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

In certain exemplary embodiments, electronic apparatus readable media comprising one or more genomic DNA sequences described herein is provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon one or more expression profiles described herein.

As used herein, the term “electronic apparatus” is intended to include any suitable computing or processing apparatus or other device configured or adapted for storing data or information. Examples of electronic apparatuses suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising one or more expression profiles described herein.

A variety of software programs and formats can be used to store the genomic DNA information of the present invention on the electronic apparatus readable medium. For example, the nucleic acid sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon one or more expression profiles described herein.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### Example I

### Protocol

The following general protocol is useful for single cell whole methylome amplification. Isolation of single cells can be performed by mouth pipetting, laser dissection, microfluidic devices, flow cytometry and the like.

In general, a single cell is lysed in lysis buffer. The transposome with a primer binding site sequence and transposition buffer are added to the cell lysis. Protease is added after the tranposition to remove the transpoase from binding to the single cell genomic DNA. Deepvent exo-DNA polymerase, dNTP, PCR reaction buffer and primers are added to the reaction mixture to fill in the gap generated from the transposon insertion. After gap repair, the DNA fragments are denatured. The resulting ssDNA with complimentary ends thus will form a stem-loop structure. To amplify the stem-loop structures, a single primer PCR reaction with step-down annealing temperature is performed. The resulting extension products are then incubated with a methylation reagent such as DNMT1. After the incubation, depending on how many rounds of methylation replication are needed, the DeepVent PCR reaction and DNMT1 incubation can be performed multiple times. The product can be directly treated with a bisulfite conversion reagent using Zymo EZ-Direct Bisulfite Kit.

The following more specific protocol for single-cell whole methylome amplification is provided.

### Single-Cell Lysis

A single cell is sorted by FACs or mouth pipiet into 2.5 ul Lysis buffer. The lysis buffer contains: 1.825 ul H₂O, 0.05 ul 1M TE buffer pH 8.0, 0.05 ul 1M KCL, 0.375 ul 0.1M DTT, 0.075 ul 10% Triton X-100, 0.125 20 mg/ml Protease Q (Qiagen). The lysis reaction happens in the following thermo-cycle: 50° C. for 20 mins, 75° C. for 20 mins, 80° C. for 5 mins. After lysis, dsDNA is released from the single cell.

### Tn5 Tagmentation

A Tn5 transposon complex is prepared for tagmentation as follows. Mix 1 ul of purified 5 uM Tn5 Protein with 1 ul 5 uM Tn5 dsDNA. Incubate at 25° C. for 45 mins. Add 98 ul of Tn5 Dilution Buffer to the 2 ul Transposon mix to achieve 0.05 uM Tn5 complex. The Tn5 Dilution Buffer includes 10 ul 1M TE Buffer pH 8.0, 4 ul 0.5M NaCl, and 84 ul H₂O. The Tn5 dsDNA includes upper strand 5′-CAT TAC GAG CGA GAT GTG TAT AAG AGA CAG-3′ (SEQ ID NO: 1) and lower strand 5′-Phos-CTG TCT CTT ATA CAC ATC invdT-3′ (SEQ ID NO: 2). To the 2.5 ul cell lysis, add 1.5 ul of 0.05 uM Tn5 transposon complex, 1 ul of 5×Tn5 Insertion Buffer. lx Buffer Condition for Tn5 Insertion Buffer includes 10 mM Tris-Hcl, 5 mM MgCl₂ at pH 7.8 at 25° C. Incubate the 5 ul reaction at 50° C. for 10 mins. Add 1 ul 1 mg/ul ProteaseQ (Qiagen) to the reaction and incubate at the following thermo-cycle: 50° C. for 20 mins and 70° C. for 30 mins. The resulting dsDNA should be 500 bp-1000 bp in length with 30 bp DNA priming sites added on both ends leaving a 9 bp gap on the 3′ end.

### Gap Repair and 1^{st} Round of PCR Reaction

To fill in the 9 bp gap, Deep vent exo-polymerase with strand displacement activity is used to repair the gap. After gap repair, the DNA fragments are heat denatured. The resulting ssDNA with complimentary ends form a stem-loop structure. To amplify the stem-loop structures, a single primer PCR reaction with step-down annealing temperature is performed. To the 6 ul reaction, add 1 ul of 10×PCR Buffer, 0.2 ul dNTP, 0.1 ul 2U/ul DeepVent exo-, 0.2 ul 1 uM 30 bp ssDNA primer having the sequence 5′-CAT TAC GAG CGA GAT GTG TAT AAG AGA CAG-3′ (SEQ ID NO: 1), 0.2 ul 100 mM MgSO₄, and 2.3 ul H₂O. 1×PCR Buffer condition includes 20 mM Tris-HCL, 10 mM KCL, 0.1% Trition X-100, pH 7.8 at 25° C. The following thermo-cycle is performed on the 10 ul reaction: 72° C. for 10 minutes, 95° C. for 3 minutes, 68° C. for 60 secs, 67° C. for 60 secs, 66° C. for 60 secs, 65° C. for 60 secs, 64° C. for 60 secs, 63° C. for 60 secs, 62° C. for 60 secs, 61° C. for 60 secs, 60° C. for 60 secs, 59° C. for 60 secs, 58° C. for 60 secs, and 72° C. for 3 minutes. This results in hemi-methylated dsDNA fragments.

### 1st Round of DNMT1 Methyl-Transfer Reaction

The resulting extension products are then incubated with DNMT. EDTA is added to chelate Mg²⁺. To the 10 ul reaction, add 1.5 ul of 10×Methyl-Transfer (MT) Buffer, 0.15 ul 160 uM SAM, 0.15 ul 100 ug/ml BSA, 0.3 ul 200 mM EDTA, 2 ul 2U/ul DNMT1, 0.9 ul H₂O. 1× buffer condition for MERLOT MT Buffer includes 20 mM Tris-HCL, 1 mM DTT, 5% Glycerol, pH 7.8 at 25° C. The 15 ul reaction is incubated at 37° C. for 3 hours. This results in methylated dsDNA fragments and a complete single round of amplification, i.e. single primer extension, and methylation.

### Multiple Rounds of Amplification and Methylation on Demand

A further 1 to 20, 1 to 10 or 1 to 5 rounds of amplification and methylation can be performed based on demand. The thermo-cycle is the same as 1^{st} round of amplification and methylation. The following reagents should be added for the 2^{nd}, 3^{rd}, 4^{th}, 5^{th}, etc., round of amplification and methylation respectively.

### 2^{nd} Round of Amplification, PCR

To the 15 ul reaction, add 1 ul of 10×PCR Buffer, 0.2 ul dNTP, 0.1 ul 2U/ul DeepVent exo-, 0.2 ul 1 uM 30 bp ssDNA primer, 0.55 ul 100 mM MgSO₄, and 2.95 ul H₂O.

### 2^{nd} Round of DNMT1 Methyl-Transfer Reaction

To the 20 ul reaction, add 1 ul of 10×Methyl-Transfer (MT) Buffer, 0.25 ul 160 uM SAM, 0.1 ul 100 ug/ml BSA, 0.325 ul 100 mM EDTA, 2 ul 2U/ul DNMT1, 0.15 ul 100 mM DTT, and 1.175 ul H₂O.

### 3^{rd} Round of Amplification, PCR

To the 25 ul reaction, add 1 ul of 10×PCR Buffer, 0.2 ul dNTP, 0.1 ul 2U/ul DeepVent exo-, 0.2 ul 1 uM 30 bp ssDNA primer, 0.85 ul 100 mM MgSO₄, and 2.65 ul H₂O.

### 3^{rd} Round of DNMT1 Methyl-Transfer Reaction

To the 30 ul reaction, add 1 ul of 10× MERLOT Methyl-Transfer (MT) Buffer, 0.35 ul 160 uM SAM, 0.1 ul 100 ug/ml BSA, 0.475 ul 200 mM EDTA, 2 ul 2U/ul DNMT1, 0.25 ul 100 mM DTT, and 0.825 ul H₂O.

### 4^{th} Round of PCR

To the 35 ul reaction, add 1 ul of 10×PCR Buffer, 0.2 ul dNTP, 0.1 ul 2U/ul DeepVent exo-, 0.2 ul 1 uM 30 bp ssDNA primer, 1.15 ul 100 mM MgSO₄, and 2.35 ul H₂O.

### 4^{th} Round of DNMT1 Methyl-Transfer Reaction

To the 40 ul reaction, add 1 ul of 10×Methyl-Transfer (MT) Buffer, 0.45 ul 160 uM SAM, 0.1 ul 100 ug/ml BSA, 0.625 ul 200 mM EDTA, 2 ul 2U/ul DNMT1, 0.35 ul 100 mM DTT, and 0.475 ul H₂O.

### 5^{th} Round of PCR

To the 45 ul reaction, add 1 ul of 10×PCR Buffer, 0.2 ul dNTP, 0.1 ul 2U/ul DeepVent exo-, 0.2 ul 1 uM 30 bp ssDNA primer, 1.45 ul 100 mM MgSO₄, and 2.05 ul H₂O.

### 5^{th} Round of DNMT1 Methyl-Transfer Reaction

To the 50 ul reaction, add 1 ul of 10×Methyl-Transfer (MT) Buffer, 0.55 ul 160 uM SAM, 0.1 ul 100 ug/ml BSA, 0.775 ul 100 mM EDTA, 2 ul 2U/ul DNMT1, 0.45 ul 100 mM DTT, and 0.125 ul H₂O.

The amplified dsDNA that has been fully methylated can be directly treated with sodium bisulfite using commercial kits such as Zymo EZ-Direct Bisulfite Kit. Bisulfite converted DNA is ready for downstream analysis such as whole genome bisulfite sequencing.

### Example II

### Kits

The materials and reagents required for the disclosed methods may be assembled together in a kit. The kits for single cell whole genome methylome sequencing of the present disclosure generally will include at least the transposome (consists of transposase enzyme and transposon DNA), nucleotides, and DNA polymerase necessary to carry out the claimed method along with primer sets as needed. The kit will also include the DNMT1 and any buffers needed, including those containing cations as described herein. The kit may also contain a chelating agent for chelating such cations during the methylation step and may also include cations for replenishing the reaction media when primer extension is being carried out. The kit will also contain directions for creating the amplified methylome from DNA samples. The kits for early cancer diagnosis of the present disclosure generally will include at least the selected sets of primers, nucleotides, and DNA polymerase necessary to carry out the claimed method. The kit will also include the DNMT1 and any buffers needed. The kit will also contain directions for amplifying targeted DNA regions from cell-free DNA samples. In each case, the kits will preferably have distinct containers for each individual reagent, enzyme or reactant. Each agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions are preferably provided with the kit.

### Example III

### Methyl Transfer Efficiency

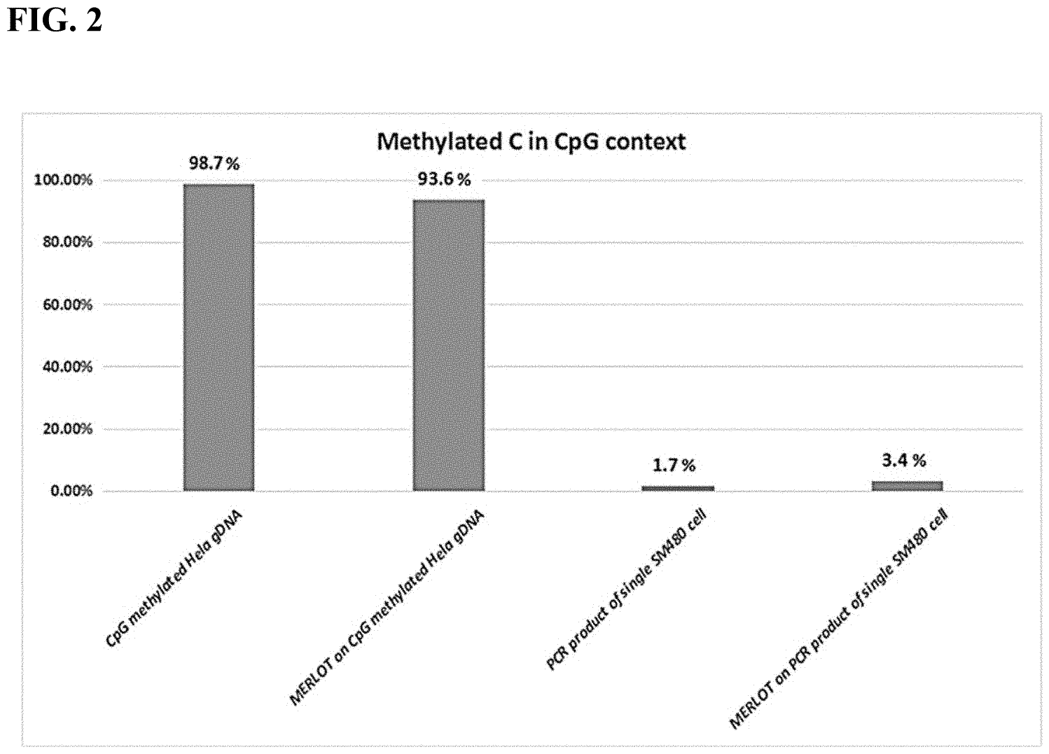

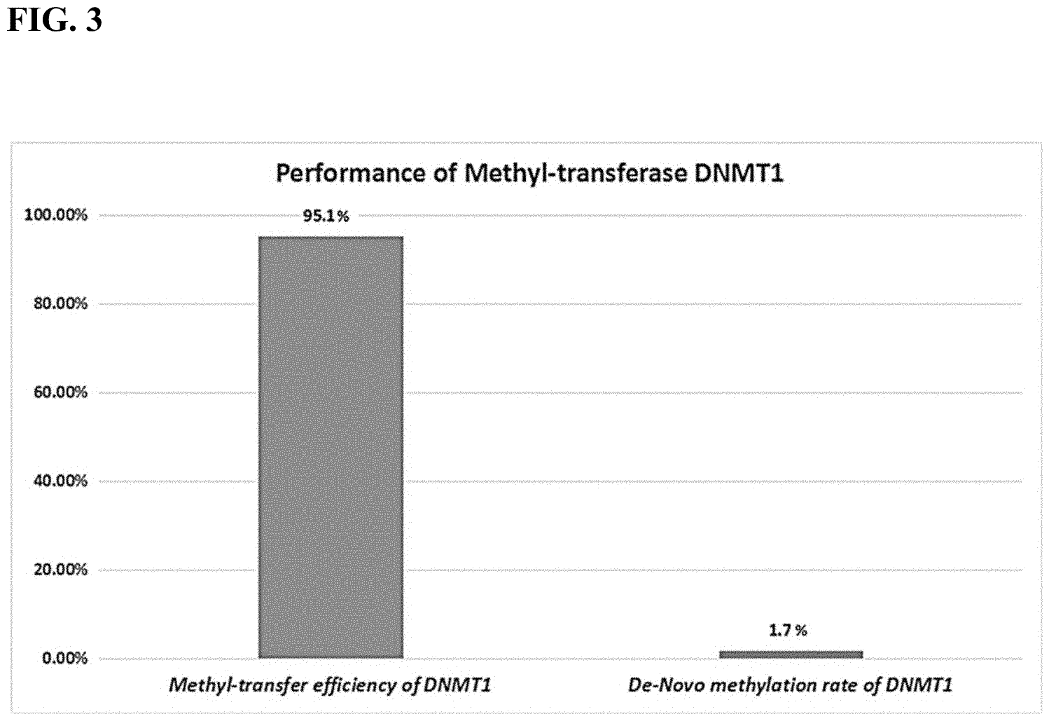

To determine the methyl-transfer efficiency of methods described herein, bisulfite sequencing (Miseq v2 chemistry kit, 2×150 bp pair end reads, 1,000,000 reads in total) was first performed on 10 pgs of fully methylated Hela gDNA and amplified DNA resulting from 1 round of single primer extension and DNMT1 incubation as described herein of 10 pg of fully methylated Hela gDNA. Among all the reads that uniquely aligned to human genome, 98.7% of the cytosine in CpG context are methylated for fully methylated Hela gDNA while 93.60% of the cytosine in CpG context are methylated for amplified DNA resulting from 1 round of single primer extension and DNMT1 incubation as described herein of 10 pg of fully methylated Hela gDNA. See FIG. 2. This indicates a methy-transfer efficiency of 95.1% during DNMT1 incubation. See FIG. 3.

DNMT1 is also known to have de-novo methylation activity. In order to infer the de-novo methylation rate of the methods described herein, bisulfite sequencing (Miseq v2 chemistry kit, 2×150 bp pair end reads, 1,000,000 reads in total) was performed on 10 pgs of PCR product of single SM480 cell gDNA and amplified DNA resulting from 1 round of single primer extension and DNMT1 incubation as described herein of 10 pg of PCR product of single SM480 cell gDNA. See FIG. 2. The result indicates a tolerable de-novo methylation rate of 1.7%. See FIG. 3.

For single cell whole methylome sequencing and without performing pre-amplification of methylome as described herein before bisulfite conversion, a single cell may be directly treated with sodium bisulfite followed by post-bisulfite amplification and sequencing. In this instance, the methylome coverage is low due to DNA lost during bisulfite conversion. Representative coverage achieved is about 20% on average for mouse methylome. See Smallwood, S. A., et al. (2014). “Single-cell genome-wide bisulfite sequencing for assessing epigenetic heterogeneity.” Nat Methods 11(8): 817-820 hereby incorporated by reference in its entirety. Also a reduced representation version is achieved with an average 4% methylome coverage. See Guo, H., et al. (2013). “Single-cell methylome landscapes of mouse embryonic stem cells and early embryos analyzed using reduced representation bisulfite sequencing.” Genome Res 23(12): 2126-2135 hereby incorporated by reference in its entirety. At this low coverage, a large amount of cells is needed to be analyzed to achieve enough statistical confidence (since for a 20% coverage, one needs to sequence about 15 cells to reproduce a methylation status of a particular CpG site for >=3 times), which renders the ability to effectively address methylation heterogeneity among cell population or analysis on rare samples such as a human embryo. Methods described herein that include single primer extension and incubation with a methyl addition agent improves the methylome coverage by 3-4 fold which greatly improves the ability to analyze rare samples and cell to cell heterogeneity.

According to one embodiment directed to cancer diagnosis, the amount of cell free DNA released from a tumor compared to plasma DNA is extremely low. To recover the methylation information of cell free tumor DNA from a large background (plasma DNA), current methods require a highly sensitive methylation detection method, for example, methylation specific qPCR which depends on mid or late stage cancer where the amount of cell free DNA from a tumor is significantly higher compared to early stage cancer. Also, such methods have poor sensitivity, i.e. around 50% for early cancer diagnosis. Instead of optimizing the methylation detection method, the methods described herein amplify the cell free DNA while maintaining the methylation status using a methylation agent and with a selected set of primers targeting differentially methylated genes. The amplification while maintaining methylation information or status provides more initial material for detection methods and increased sensitivity.

According to one aspect, methyl transfer efficiency and de novo methylation rate are utilized to maximize efficiency of making an amplified methylome. Methyl-transfer efficiency refers to the percentage of hemi-methylated CpGs that become fully methylated after incubation with a methylation agent such as DNMT1. De-Novo Methylation rate refers to the percentage of non-methylated CpGs that become fully methylated after incubation.

A 100% methyl-transfer efficiency means a perfect replication of the methylation status of the original hemi-methylated CpG sites of the template. A 95% methyl-transfer efficiency means in 1 round of DNMT1 incubation, 5% of the methylation status will be randomly lost and a 5% false-negative rate for methylation status analysis is introduced. For 3 rounds of amplification, i.e. 3 rounds of single primer extension, and incubation with a methylation agent, the false-negative rate increases exponentially, as 1-0.95³=14.3%. Although when calculating the methylation of a particular gene, the methylation status of multiple CpG sites is taken into account so as to reduce the false-negative rate and methyl-transfer efficiency is preferred to be at least 95%.

A 0% De-Novo Methylation rate means no methyl group is added to the unmethylated CpG sites of the original template. A 2% De-Novo Methylation rate means in 1 round of DNMT1 incubation, 2% of the CpG sites that were not methylated will be randomly methylated and a 2% false-positive rate for methylation status analysis is introduced. For 3 rounds of amplification, i.e. 3 rounds of single primer extension, and incubation with a methylation agent, the false-positive rate increases linearly, as 3×0.02=6%. Although when calculating the methylation of a particular gene, the methylation status of multiple CpG sites is taken into account so as to reduce the false-positive rate and De-Novo Methylation is preferred to be no more than 2%.

### Example IV

### Methods of Diagnosis Based on Methylation Analysis

Aspects of the present disclosure are based on the diagnosis or prognosis of a condition such as cancer. According to one aspect, a blood sample including cell free DNA is obtained from an individual. The cell free DNA is processed according to the methods described herein and analyzed to determine the presence of cancer cell DNA based on identifying a methylation pattern corresponding to cancer cell DNA. By amplifying the methylation status of targeted differential methylated loci, the sensitivity of capturing low abundance cell free tumor DNA is increased. The method includes (a) fragmenting a double stranded DNA sequence obtained from a blood sample from the individual, wherein the double stranded DNA sequence has a methylation pattern, to produce fragment template double stranded DNA sequences having a methylation pattern and including a primer binding site on each 5′ end of the fragment template double stranded DNA sequences, (b) separating the fragment template double stranded DNA sequences into upper and lower template strands, (c) extending the upper and lower template strands using a primer, a polymerase and nucleotides to produce non-methylated complementary strands resulting in hemi-methylated double stranded DNA sequences corresponding to the fragment template double stranded DNA sequences having a methylation pattern, (d) treating the hemi-methylated double stranded DNA sequences to add methyl groups at positions corresponding to methylated cytosine in the corresponding fragment template double stranded DNA sequences to produce fully methylated fragment template double stranded DNA sequences; repeating steps (b) to (d) from 1 to 5 times to produce fully methylated amplicons of the fragment template double stranded DNA sequences; treating the fully methylated amplicons of the fragment template double stranded DNA sequences with a reagent to convert cytosine residues to uracil; determining methylated cytosine pattern; comparing the methylated cytosine pattern to a standard methylated cytosine pattern for cancer DNA; and diagnosing the individual with cancer when the determined methylated cytosine pattern matches the standard methylated cytosine pattern for cancer DNA.

According to one aspect, methods are described that include (a) extracting cell free DNA or genomic DNA that may contain cell free tumor DNA from a liquid biopsy from the individual patient, wherein the cell free tumor DNA sequence has a different methylation pattern compare with normal somatic cells; (b) separating the double stranded DNA sequences into upper and lower template strands; (c) extending the upper and lower template strands using a polymerase, nucleotides and selected sets of primers targeting differential methylated loci to produce non-methylated complementary strands resulting in hemi-methylated double stranded DNA sequences for selected differential methylated loci; (d) adding EDTA to chelate magnesium ions in an equal molar fashion to create a non-magnesium buffer condition; (e) treating the hemi-methylated double stranded DNA sequences with methyl-transferase to add methyl groups at positions corresponding to methylated cytosine in the corresponding double stranded DNA sequences to produce fully methylated double stranded DNA sequences of selected differential methylated loci; (f) after such treatment, adding magnesium ion to an ideal concentration for the subsequent primer extension reaction, such as using PCR conditions as needed; and (g) repeating the steps to produce fully methylated amplicons of the selected differential methylated loci. Then, the fully methylated amplicons may be treated with a reagent to convert cytosine residues to uracil while keeping methylated cytosine residues unchanged. The methylated cytosine pattern may be determined.

Whether cell free tumor DNA was present in the sample is determined by comparing the methylated cytosine pattern of cell free DNA with the methylated cytosine pattern of different cancer tissue. The individual may be diagnosed with a certain type of cancer when the determined methylated cytosine pattern contains a standard methylated cytosine pattern for a certain type of cancer.

According to one aspect, plasma DNA is extracted from a blood sample. A selected set of biotin attached PCR primers which targets target loci of differentially methylated genes in cancer is used to perform 1 round of PCR primer extension and incubation with a methylation agent to create hemi-methylated amplicons of the target loci. DNMT1 may be used for the methyl-transfer reaction to create methylated amplicons of the targeted gene loci. The primer extension and the incubation step may be repeated as desired to produce the amplified methylome of the selected target loci. The primer extension and the incubation step may be repeated from 1 to 20 times or greater as desired to produce the amplified methylome of the selected target loci. The biotin-labeled amplicons are isolated or “pulled down” using for example an avidin binding partner attached to a substrate. The isolated amplicons are treated with sodium bisulfite. The bisulfite converted version of the same set of PCR primers is used to amplify the bisulfite converted amplicons. The same PCR primers cannot amplify the amplicons treated with bisulfite since all unmodified cytosines are converted to uracil and so one has to change the primers slightly to match the C-U conversion. This step is followed by library preparation and sequencing. Alternatively, this step is followed by methylation specific qPCR to probe the methylation status of the bisulfite treated amplicons, in one embodiment, a single gene at a time. An exemplary gene is a cancer gene such as SEPT9. Suitable methylation specific qPCR is described in Jorja D Warren et al. Septin 9 methylated DNA is a sensitive and specific blood test for colorectal cancer. BMC Medicine 2011, 9:133 hereby incorporated by reference in its entirety. According to one aspect, the amplification to maintain methylation status as described herein to produce an amplified methylome improves sensitivity beyond known qPCR techniques to directly probe methylation status of cancer genes of cell free DNA, such as is carried out by EpiProColon.

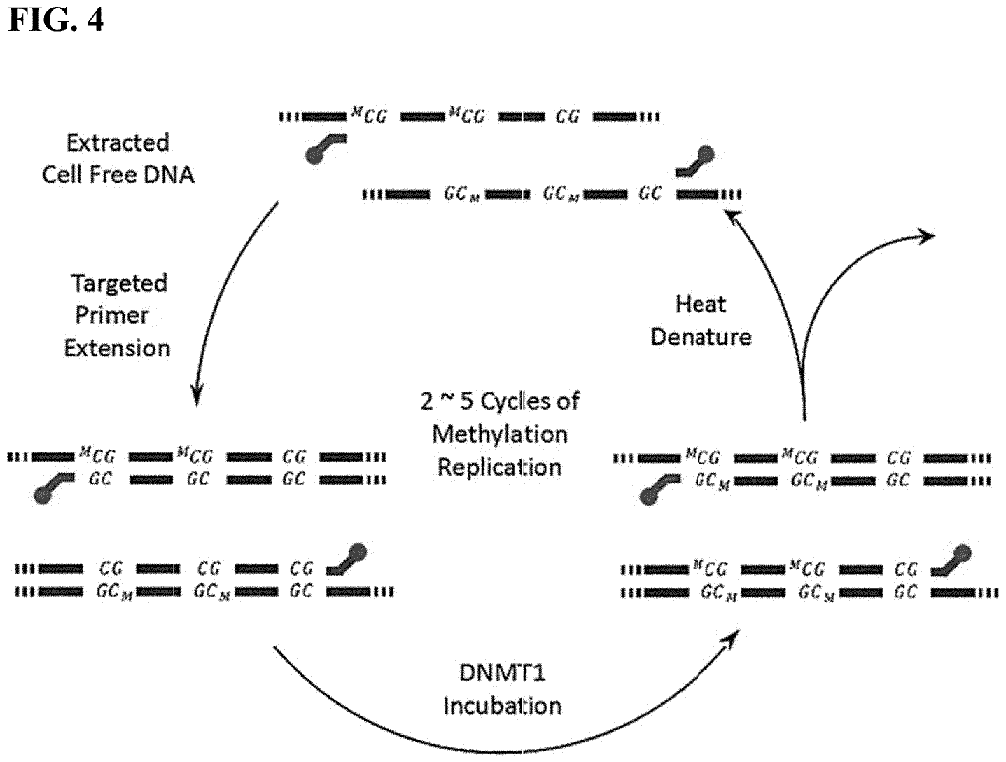

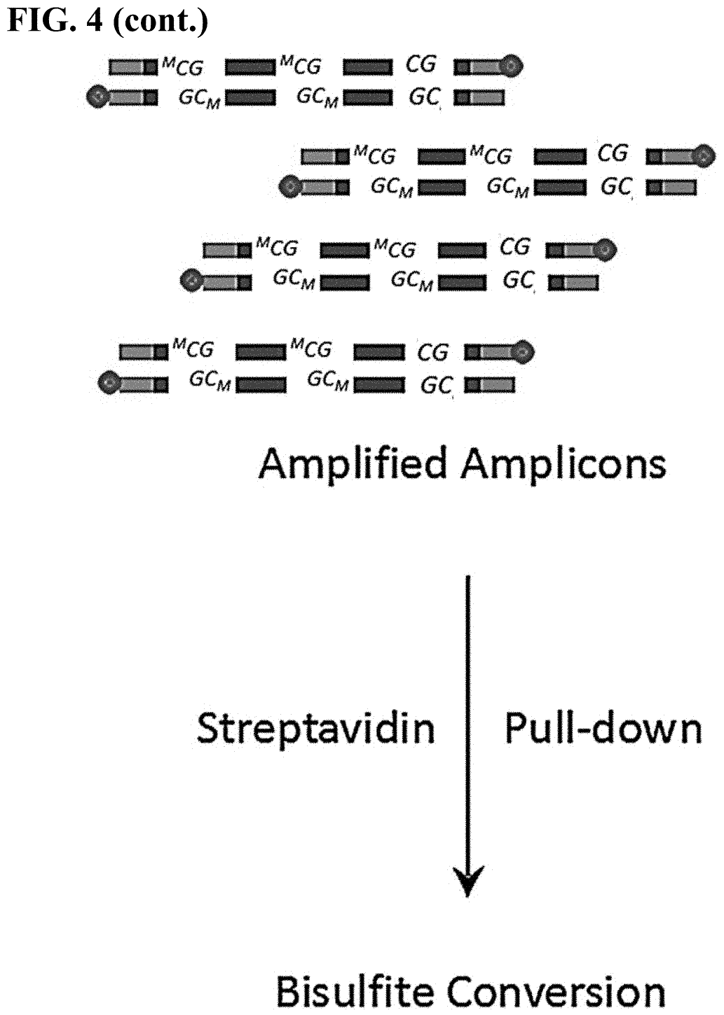

More specifically, the amplification and methylation methods can be carried out for methylation analysis of cell free tumor DNA as shown in schematic in FIG. 4. Cell free DNA extraction from blood plasma is carried out as follows. 10 ml of blood was collected in an EDTA (ethylenediaminetetraacetic acid) vacutainer tube. Each tube was centrifuged for 12 minutes at 1350×g±150×g at room temperature. Plasma was transferred without disturbing the buffy coat to a clean 15 ml conical tube. The sample was centrifuged a second time for 12 minutes at 1350×g±150×g. Plasma was transferred without disturbing the pellet to a 4 ml tube. Cell free DNA was extracted from 4 ml of plasma using QIAamp Circulating Nucleic Acid Kit (QIAGEN) and eluted in 50 ul elution buffer. The extraction of Cell free DNA can also be done by Quick-cfDNA™ Serum & Plasma Kit (ZYMO), Chamagic cfDNA 5k Kit (Chemagen), NucleoSpin Plasma XS (TaKaRa) etc. The 50 ul eluted DNA is further concentrated by DNA concentrate and clean Kit (ZYMO) and eluted in 6 ul Elution buffer into a PCR tube.

Primer extension and methylation resulting in an amplified methylome is carried out on extracted cell free DNA using a set of PCR primers targeting differential methylated gene loci. Differential methylated genes, which are genes that have a different methylation status in a cancer cell compare to a normal somatic cell, are selected based on standard cancer methylation data. The differential methylated genes include but are not limited to: SEPT9; TMEM106A; NCS1; UXS1; HORMAD2; REC8; DOCK8; CDKL5; SNRPN; SNURF; ABCC6; CA10; DBC2; HEPACAM; KRT13; MYO3A; NKX6-2; PMF1; POU4F2; SYNPO2/myopodin; ZNF154; 30ST3B; ACADL; ATOH1/hATH; BECN1; C14; CBFA2T3; COL7A1; CREBBP; CXCL1; EDN3; ETS1; FAM110A/c20; FAM19A4/FLJ25161; FAT4; FGFR4; FOXC1; FOXF1; GHSR; GJB2/CX26; GPR180/ITR; HDAC1; HSD17B1; HSD17B2; HSD17B4; IPF1; ISL1; ITIH5; LEBREL1/P3H2; LEBREL2/P3H3; LRRC49; MGA; miR-124; miR-196a-2; miR-335; ADAMTS5; MYBL2; NFIX; NRN1; OGG1; PCDHGB6; PPP2R2B; PRDM12; PTRF; RNF20; ST18; STK36; STMN1; SULT1A1; SYNM; THAP10; TOX; TSC1; UAP1L1; UGT3A1; ZBTB8A; ZNF432; ADAMTS12; ADHFE1; BARX1; BEND4; CASR; CD109; CDX1; CNR1/CB(1) receptor; CNRIP1; CNTFR; DEXI; DUSP26; EDIL3; ELMO1; EXTL3; EYA2; FLT1; GJC1; GLP1R; GPR101; GRIN2/NMDAR2A; GSPT2; HOMER2; INA; KCNK12; LAMA1; LRP2/megalin; KISS1; MBD4/MED1; MCC; miR-342; miR-345; NDRG4; NGFR; NR3C1/GR; PIK3CG; PPARG; PTGIS; PTPRR; QKI; RGMA; SEPT9; SPG20; STARD8; STOX2; TBX5; THBS4/TSP4; TMEM8B/NGX6; VSX2/HOX10; ANGTL2; AXIN1; CCBE1; CTGF/IGFBP8; DNAJC15; FBXO32; FILIPIL; FZD4; GPR150; GUCY2C; HOXB5; ITGA8; LRP5; miR-130b; NFATX; PTPRN; RUNX1T1; TERC/hTR; TES; TMCO5; IFFO1; ALK; CHGA; CSMD2; DES; DUSP6; ELOVL4; FANCG; FGF2; FGF3; FGF5; FGF8; FGFR1; FLT3; FLT4; GAS1; GEMIN2/SIP1; HIC2; HSD17B12; IGFBP5; ITPR2; LMO1/RBTN1; I-mfa; miR-132; NEFL; NKX2-8; NTRK3/TRKC; NTSR1; PRG2; PTCH2; SLC32A1; TRH; TUBB3; ZNF415; CLSTN1; HIST1H4K; HIST2H2BF; INHA/inhibin alpha; KCNMA; NKX3.1; NPBWR1/GPR7; NSMCE1/NSE1; PXMP4/PMP24; RGS2; S100A6; SLC18A2; SPRY4; SVIL; TFAP2E; TGFB2; ZNF132; NFATC; CST6; MDFI; ADAM23; ALDH1A3; APC; BNC1; BRCA1; CADM1/TSLC1/IGSF4; CASB; CAV1; CCNA1; CCND2; CD2/SRBC; CD44; CDH1/E-cadherin; CDH13/H-cadherin; CDKN1C/KIP2/p57; CDKN2A/ARF/p14; CDKN2B/INK4B/p15; CHFR; CIDEA; CLSTN1; COL1A2; CYP1A1; DAB2IP; DAPK1; DBC1; DIRAS(3)/ARHI; DKK3; DLC1; DLEC1; DPYS; EOMES; EPHA5; ESR1/ER-alpha; ESR2/ER-beta; FHIT; FHL1; GAS7; GATA5; GSTP1; HIC1; HISTIH4K; HIST2H2Bf; HOXA11; HOXA9; HS3ST2/30ST2; ID4; IGF2; IGFBP3; KCNMA1; LAMA3; LAMC2; MAL; MARVELD1; MDFI; MGMT; MINT1/APBA1; MINT2/APBA2; MINT31; miR-34a; miR-34b; miR-34c; miR-9-1; MLH1; MMP2; MSH2; MSX1; MYOD1/MYF-3; NID2; NKX3-1; NPBWR1; NSMCE1/NSE1; OPCML; p14; PCDH17; PDLIM4/RIL; PENK; PGR; PITX2; PLAU/uPA; PRDM2/RIZ1; PTEN/MMAC1; PTGS2/COX2; PXMP4/PMP24; PYCARD/ASC/TMS1; RARB; RARB2; RARRES1/TIG1; RASSF1; RASSF1A; RASSF2; RB1; RBP1/CRBP1; RGS2; RPIA; RPRM/Reprimo; RUNX3; S100A6; SCGB3A1/HIN1; SERPINB5/maspin; SFN/14-3-3 sigma; SFRP1/SARP2; SFRP2; SFRP4; SFRP5; SLC18A2; SLC5A8; SLIT2; SOCS1; SOX11; SOX17; SPARC; SPOCK2; SPRY4; STK11/LKB1; SVIL; SYK; TCF21; TERT; TFAP2E; TFPI2; TGFB2; THBS1; TIMP3; TMEFF2/HPP1/TPEF; TNFRSF10C/DcR1; TNFRSF10D/DcR2; TNFRSF25/DR3; TWIST1; UCHL1/PGP9.5; VIM; WIFI; WWOX; XAF1; ZNF132

According to one exemplary embodiments, the primer mix is an equal mix of 21 pairs of biotin modified primers that each targets a single differential methylated gene including SEPT9; TMEM106A; NCS1; UXS1; HORMAD2; REC8; DOCK8; CDKL5; SNRPN; SNURF; ABCC6; CA10; DBC2; HEPACAM; KRT13; MYO3A; NKX6-2; PMF1; POU4F2; SYNPO2/myopodin; and CDH1/E-cadherin. The combination is selected because the different combination of the methylation status of the 21 targeted genes covers the diagnosis of 6 types of common cancer including: brca: Breast Invasive Carcinoma; coad: Colon Adenocarcinoma; lihc: Liver Hepatocellular Carcinoma; prad: Prostate Adenocarcinoma; stad: Stomach Adenocarcinoma; and ucec: Uterine Corpus Endometrial Carcinoma. One of skill in the art will understand that other cancers can be diagnosed using combinations of other cancer genes and their methylation status or characteristics. Additional methods may include more primers or change the composition of the primer mix to cover more types of cancer or increase cancer diagnosis sensitivity and specificity. Methods can be carried out as follows.

### 1^{st} Round of PCR Reaction Using Primer Mix

To the 6 ul elution, add 1 ul of 10×PCR Buffer, 0.2 ul dNTP, 0.1 ul 2U/ul DeepVent exo-, 0.2 ul 1 uM MERLOT biotin-primer mix, 0.2 ul 100 mM MgSO₄, and 2.3 ul H₂O. The 1× MERLOT PCR Buffer condition includes 20 mM Tris-HCL, 10 mM KCL, 0.1% Trition X-100, pH 7.8 at 25° C. The following thermo-cycle is performed on the 10 ul reaction to span all annealing temperature for 21 primers (58° C. to 64° C.): 94° C. for 2 minutes, 64° C. for 60 secs, 63° C. for 60 secs, 62° C. for 60 secs, 61° C. for 60 secs, 60° C. for 60 secs, 59° C. for 60 secs, 58° C. for 60 secs, and 72° C. for 3 minutes. This results in hemi-methylated dsDNA fragments where one strand is attached with biotin.

### 1^{st} Round of DNMT1 Methyl-Transfer Reaction

To the 10 ul reaction, add 1.5 ul of 10×MERLOT Methyl-Transfer (MT) Buffer, 0.15 ul 160 uM SAM, 0.15 ul 100 ug/ml BSA, 0.3 ul 200 mM EDTA, 2 ul 2U/ul DNMT1, and 0.9 ul H₂O. The 1× buffer condition for MT Buffer includes 20 mM Tris-HCL, 1 mM DTT, 5% Glycerol, pH 7.8 at 25° C. The 15 ul reaction is incubated at 37° C. for 3 hours. This results in methylated dsDNA fragments and a complete single round of amplification and methylation.

### Multiple Rounds of MERLOT on Demand

A further 1 to 4 rounds of amplification and methylation can be performed based on demand. The thermo-cycle is the same as 1^{st} round of amplification and methylation. The following reagents should be added for the 2^{nd}, 3^{rd}, 4^{th}, 5^{th} round of amplification and methylation respectively.

### 2^{nd} Round of PCR

To the 15 ul reaction, add 1 ul of 10×PCR Buffer, 0.2 ul dNTP, 0.1 ul 2U/ul DeepVent exo-, 0.2 ul 1 uM MERLOT biotin-primer mix, 0.55 ul 100 mM MgSO₄, and 2.95 ul H₂O.

### 2^{nd} Round of DNMT1 Methyl-Transfer Reaction

To the 20 ul reaction, add 1 ul of 10×Methyl-Transfer (MT) Buffer, 0.25 ul 160 uM SAM, 0.1 ul 100 ug/ml BSA, 0.325 ul 100 mM EDTA, 2 ul 2U/ul DNMT1, 0.15 ul 100 mM DTT, and 1.175 ul H₂O.

### 3^{rd} Round of PCR

To the 25 ul reaction, add 1 ul of 10×MERLOT PCR Buffer, 0.2 ul dNTP, 0.1 ul 2U/ul DeepVent exo-, 0.2 ul 1 uM MERLOT biotin-primer mix, 0.85 ul 100 mM MgSO₄, 2.65 ul H₂O.

### 3^{rd} Round of DNMT1 Methyl-Transfer Reaction

To the 30 ul reaction, add 1 ul of 10×Methyl-Transfer (MT) Buffer, 0.35 ul 160 uM SAM, 0.1 ul 100 ug/ml BSA, 0.475 ul 200 mM EDTA, 2 ul 2U/ul DNMT1, 0.25 ul 100 mM DTT, and 0.825 ul H₂O.

### 4^{th} Round of PCR

To the 35 ul reaction, add 1 ul of 10×PCR Buffer, 0.2 ul dNTP, 0.11 ul 2U/ul DeepVent exo-, 0.2 ul 1 uM MERLOT biotin-primer mix, 1.15 ul 100 mM MgSO₄, and 2.35 ul H₂O.

### 4^{th} Round of DNMT1 Methyl-Transfer Reaction

To the 40 ul reaction, add 1 ul of 10×Methyl-Transfer (MT) Buffer, 0.45 ul 160 uM SAM, 0.1 ul 100 ug/ml BSA, 0.625 ul 200 mM EDTA, 2 ul 2U/ul DNMT1, 0.35 ul 100 mM DTT, and 0.475 ul H₂O.

### 5^{th} Round of PCR

To the 45 ul reaction, add 1 ul of 10×PCR Buffer, 0.2 ul dNTP, 0.1 ul 2U/ul DeepVent exo-, 0.2 ul 1 uM MERLOT biotin-primer mix, 1.45 ul 100 mM MgSO₄, and 2.05 ul H₂O.

### 5^{th} Round of DNMT1 Methyl-Transfer Reaction

To the 50 ul reaction, add 1 ul of 10×Methyl-Transfer (MT) Buffer, 0.55 ul 160 uM SAM, 0.1 ul 100 ug/ml BSA, 0.775 ul 100 mM EDTA, 2 ul 2U/ul DNMT1, 0.45 ul 100 mM DTT, 0.125 ul H₂O.

### Enrichment Using Dynabeads and Bisulfite Conversion

The amplified and methylated dsDNA contains a biotin molecule attached to both ends of the DNA amplicons. The amplicons are enriched by standard Dynabeads M-280 Streptavidin wash and eluted in 20 ul Elution buffer. The amplified differential methylated gene amplicons are treated with sodium bisulfite following the directions of Zymo EZ-Direct Bisulfite Kit. Bisulfite converted DNA is ready for downstream analysis such as Methylation specific qPCR, NGS sequencing, Pyro-Sequencing, Sanger Sequencing, etc. The methylation status of the gene amplicons are compared with the known methylation status of cancer genes, i.e. a standard, to determine through a match, i.e. methylation status similar to known methylation status of cancer cell DNA, whether nucleic acids from a cancer cell are present in the initial sample tested.

### Example V

### Amplification and Methylation in Synthetic Templates

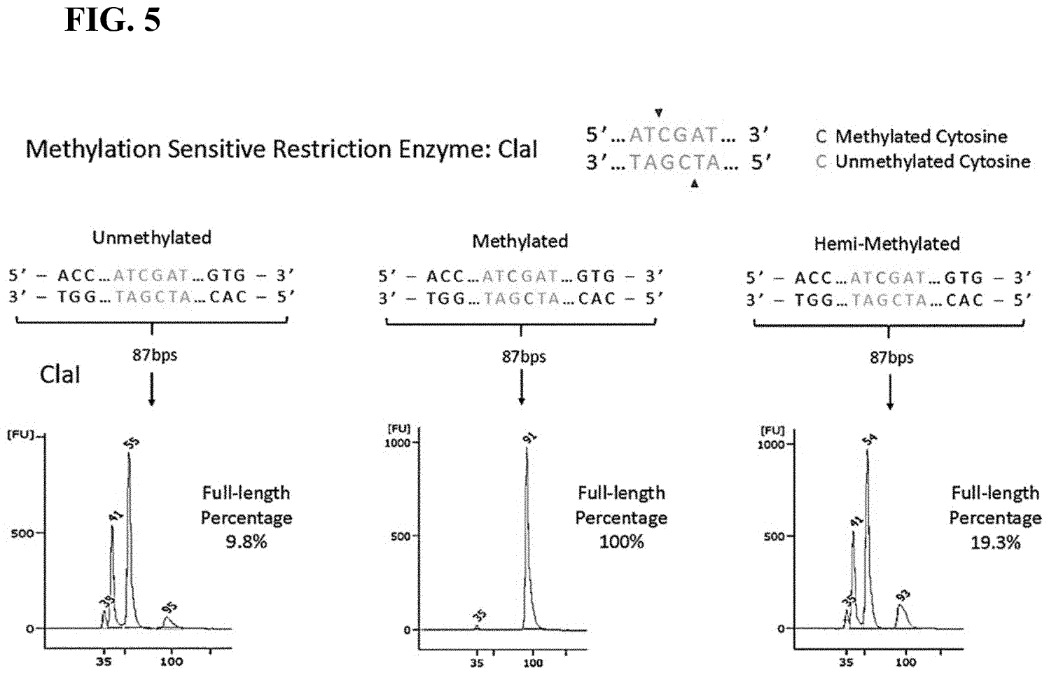

To test the performance of DNMT1 in vitro and to develop an optimal reaction buffer for the amplification and methylation reactions described herein, synthetic dsDNA which contains a methylation sensitive restriction cutting site is used as shown FIG. 5. The 87 bp dsDNA contains a 6 bp ClaI motif, 5′-ATCGAT-3′. The 87 bp dsDNA will be cleaved into two fragments once incubated with Clai if the CpG site is unmethylated. If the CpG site is methylated, the dsDNA will not be cleaved. When only one strand of the dsDNA contains a methylated Cytosine, Clai's cleavage rate is largely reduced but will still result in fragmentation if the reaction time is long enough to saturation. By running an electrophoresis on a bioanalyzer after Clai incubation for 3 hours (saturation), the exact percentage of intact dsDNA template is calculated which indicates the percentage of methylated template in the sample.

The 87 bp dsDNA template is:

Upper Strand:
(SEQ ID NO: 3)
5′-ACC TGT GAC TGA GAC ATC TGA AGG TGC AAT CAG GTG
TCA GTC TTA AAG GAT CGA TAA GGA AGC GGA AGT AGT
GGT CTC GTC GTA GTG-3′
Lower Strand:
(SEQ ID NO: 4)
5′-CAC TAC GAC GAG ACC ACT ACT TCC GCT TCC TTA TCG
ATC CTT TAA GAC TGA CAC CTG ATT GCA CCT TCA GAT
GTC TCA GTC ACA GGT-3′.

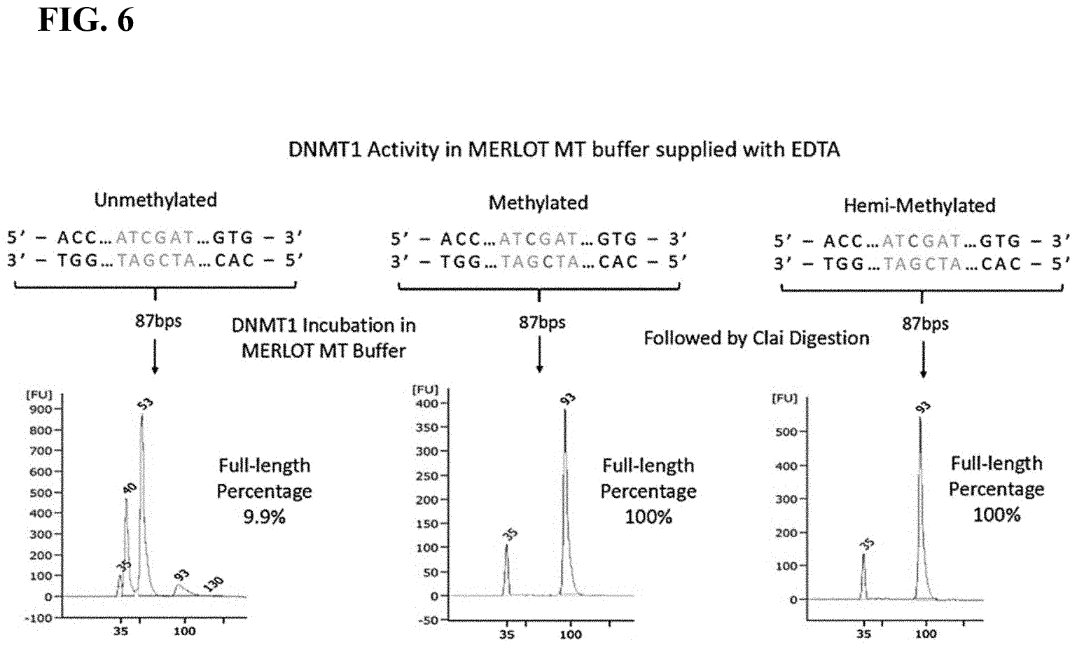

To estimate the methyl-transfer efficiency of DNMT1 in certain buffer conditions, hemi-methylated dsDNA template is incubated with DNMT1 in homemade buffer followed by Clai cleavage and electrophoresis as shown in FIG. 6. DNMT1 could achieve near 100% methyl-transfer efficiency in a 15 ul reaction containing 2 ul 2U/ul DNMT1 (NEB). The reaction buffer condition is: 1×MT buffer supplied with 0.15 ul 160 uM SAM, 0.15 ul 100 ug/ml BSA.

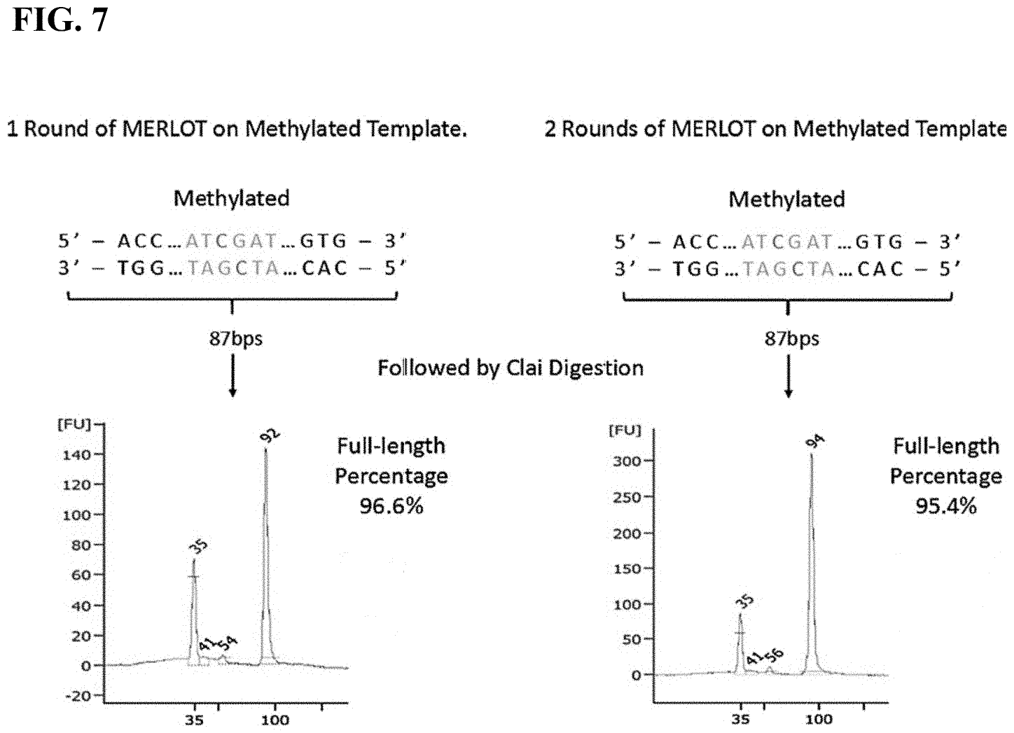

To estimate the de novo methylation rate of DNMT1 in certain buffer conditions, unmethylated dsDNA template is incubated with DNMT1 in homemade buffer followed by Clai cleavage and electrophoresis as shown in FIG. 7. DNMT1 displays near 0.1% de novo methylation rate in a 15 ul reaction incubated with 2 ul 2U/ul DNMT1 (NEB) for 3 hours. The reaction buffer condition is: 1×MT buffer supplied with 0.15 ul 160 uM SAM, 0.15 ul 100 ug/ml BSA, 0.3 ul 200 mM EDTA. 1× buffer condition for MT Buffer includes 20 mM Tris-HCL, 1 mM DTT, 5% Glycerol, pH 7.8 at 25° C. DNMT1's robust performance can be achieved when reacting in MT buffer, but in order to amplify the methylation status, a robust polymerase extension reaction is also needed. The ideal buffer condition for polymerase extension should not conflict with MT buffer and the switching between PCR buffer and MT buffer should not be complicated. Currently, amplification and methylation reactions described herein perform polymerase extension using 0.1 ul 2U/ul DeepVent exo- in a 10 ul reaction volume. The buffer condition is 1×PCR Buffer supplied with 0.2 ul dNTP and 0.2 ul 100 mM MgSO₄. The buffer switching is achieved by chelating Mg2+ with EDTA. 1×PCR Buffer condition includes 20 mM Tris-HCL, 1 mM DTT, 5% Glycerol, pH 7.8 at 25° C. The thermo cycle for polymerase extension of 87 bp dsDNA template is 94° C. for 2 minutes, 58° C. for 60 secs, and 72° C. for 3 minutes. The forward primer is 5′-ACC TGT GAC TGA GAC ATC TG-3′ (SEQ ID NO: 5). The reverse primer is 5′-CAC TAC GAC GAG ACC ACT AC-3′ (SEQ ID NO: 6).

By combining polymerase extension and DNMT1 methyl-transfer reaction (MERLOT method), one can achieve the replication of the methylation status of the original template. 1 Round of MERLOT method on the 87 bp methylated template followed by Clai cleavage and electrophoresis results in 96.6% full-length template, which indicates a 96.6% methyl-transfer efficiency of DNMT1. 2 Rounds of MERLOT on the 87 bp methylated template followed by Clai cleavage and electrophoresis results in 95.4% full-length template, indicating a success buffer switching using chelation reaction.

### Example VI

### Embodiments

The present disclosure provides a method a method of making an amplified methylome including (a) fragmenting a double stranded DNA sequence having a methylation pattern to produce fragment template double stranded DNA sequences having a methylation pattern and including a primer binding site on each 5′ end and 3′ end of the fragment template double stranded DNA sequences, (b) separating the fragment template double stranded DNA sequences into upper and lower template strands, (c) extending the upper and lower template strands using primers, a polymerase and nucleotides to produce non-methylated complementary strands resulting in hemi-methylated double stranded DNA sequences corresponding to the fragment template double stranded DNA sequences having a methylation pattern, (d) treating the hemi-methylated double stranded DNA sequences with methyl transferase and a source of methyl groups to add methyl groups at positions corresponding to methylated cytosine in the corresponding fragment template double stranded DNA sequences to produce fully methylated fragment template double stranded DNA sequences; and (e) repeating steps (b) to (d) to produce fully methylated amplicons of the fragment template double stranded DNA sequences. According to one aspect, the method further includes treating the fully methylated amplicons of the fragment template double stranded DNA sequences with a reagent to convert cytosine residues to uracil and analyzing methylated cytosine pattern. According to one aspect, the fragmenting in step (a) results from contacting the double stranded DNA sequence with a library of transposomes with each transposome of the library having its own unique associated barcode sequence, wherein each transposome of the library includes a transposase and a transposon DNA homo dimer, wherein each transposon DNA of the homo dimer includes a transposase binding site, a unique barcode sequence and a primer binding site, wherein the library of transposomes bind to target locations along the double stranded DNA sequence and the transposase cleaves the double stranded DNA sequence into the fragment template double stranded DNA sequences, with each fragment template double stranded DNA sequence including one member of a unique barcode sequence pair on each end of the fragmente template double stranded DNA sequence, gap filling a gap between the transposon DNA and the fragment template double stranded DNA sequence to form a library of fragment template double stranded DNA sequences having primer binding sites at each end. According to one aspect, step (c) includes magnesium ions and the treating of step (d) includes adding a chelating agent to chelate magnesium ions. According to one aspect, step (c) includes magnesium ions and the treating of step (d) includes adding EDTA to chelate magnesium ions. According to one aspect, step (c) includes magnesium ions and the treating of step (d) includes adding EDTA to chelate magnesium ions in an equal molar fashion to create an ideal buffer condition for methyl-transferase. According to one aspect, step (e) includes adding magnesium ion in repeated step (c) to create an ideal primer extension buffer condition for primer extension. According to one aspect, the methyl transferase is DNMT1. According to one aspect, the transposase is Tn5 transposase, Mu transposase, Tn7 transposase or IS5 transposase. According to one aspect, the reagent to convert cytosine residues to uracil is sodium bisulfite. According to one aspect, the double stranded DNA sequence is genomic DNA. According to one aspect, the double stranded DNA sequence is whole genomic DNA obtained from a single cell or is cell free DNA. According to one aspect, the double stranded DNA sequence is genomic DNA from a prenatal cell, a cancer cell, or a circulating tumor cell. According to one aspect, the double stranded DNA sequence is cell free tumor cell genomic DNA obtained from a blood sample from an individual. According to one aspect, steps (b) to (d) are repeated between 1 to 20 times. According to one aspect, steps (b) to (d) are repeated between 1 to 10 times. According to one aspect, steps (b) to (d) are repeated between 1 to 5 times. According to one aspect, the fully methylated amplicons of the fragment template double stranded DNA sequences are treated with a reagent to convert cytosine residues to uracil. According to one aspect, the reagent to convert cytosine residues to uracil is an enzyme of the family APOBEC. According to one aspect, the reagent to convert cytosine residues to uracil is APOBEC3A. According to one aspect, the primers are loci specific primers. According to one aspect, the primers are disease specific primers. According to one aspect, the primers are cancer specific primers.

The present disclosure provides a method of diagnosing an individual with cancer including (a) fragmenting a double stranded DNA sequence obtained from a liquid biopsy sample from the individual, wherein the double stranded DNA sequence has a methylation pattern, to produce fragment template double stranded DNA sequences having a methylation pattern and including a primer binding site on each 5′ end and 3′ end of the fragment template double stranded DNA sequences, (b) separating the fragment template double stranded DNA sequences into upper and lower template strands, (c) extending the upper and lower template strands using cancer specific primers, a polymerase and nucleotides to produce non-methylated complementary strands resulting in hemi-methylated double stranded DNA sequences corresponding to the fragment template double stranded DNA sequences having a methylation pattern, (d) treating the hemi-methylated double stranded DNA sequences with methyl transferase and a source of methyl groups to add methyl groups at positions corresponding to methylated cytosine in the corresponding fragment template double stranded DNA sequences to produce fully methylated fragment template double stranded DNA sequences; (e) repeating steps (b) to (d) to produce fully methylated amplicons of the fragment template double stranded DNA sequences; treating the fully methylated amplicons of the fragment template double stranded DNA sequences with a reagent to convert cytosine residues to uracil; determining methylated cytosine pattern; comparing the methylated cytosine pattern to a standard methylated cytosine pattern for cancer DNA; determining differences between the methylated cytosine pattern and the standard methylated cytosine pattern for cancer DNA; and diagnosing the individual with cancer when the determined methylated cytosine pattern matches the standard methylated cytosine pattern for cancer DNA. According to one aspect, the liquid biopsy sample is a blood sample, spinal fluid sample or urine sample. According to one aspect, step (c) includes magnesium ions and the treating of step (d) includes adding a chelating agent to chelate magnesium ions. According to one aspect, step (c) includes magnesium ions and the treating of step (d) includes adding EDTA to chelate magnesium ions. According to one aspect, step (c) includes magnesium ions and the treating of step (d) includes adding EDTA to chelate magnesium ions in an equal molar fashion to create an ideal buffer condition for methyl-transferase. According to one aspect, step (e) includes adding magnesium ion in repeated step (c) to create an ideal primer extension buffer condition for primer extension. According to one aspect, the methyl transferase is DNMT1. According to one aspect, the reagent to convert cytosine residues to uracil is sodium bisulfite. According to one aspect, the reagent to convert cytosine residues to uracil is an enzyme of the family APOBEC. According to one aspect, the reagent to convert cytosine residues to uracil is APOBEC3A. According to one aspect, the double stranded DNA sequence is whole genomic DNA obtained from a single cell. According to one aspect, the double stranded DNA sequence is genomic DNA from cancer cell or a circulating tumor cell. According to one aspect, the double stranded DNA sequence is cell free tumor cell genomic DNA obtained from a blood sample from an individual. According to one aspect, steps (b) to (d) are repeated between 1 to 20 times. According to one aspect, steps (b) to (d) are repeated between 1 to 10 times. According to one aspect, steps (b) to (d) are repeated between 1 to 5 times. According to one aspect, the primers are cancer specific primers. According to one aspect, determining methylated cytosine patterns includes Next-generation sequencing, methylation specific qPCR, or a methylation detecting micro-array. According to one aspect, the cancer is a member selected from the group consisting of breast invasive carcinoma, colon adenocarcinoma, liver hepatocellular carcinoma, prostate adenocarcinoma, stomach adenocarcinoma, and uterine corpus endometrial carcinoma.

The disclosure provides a method of early cancer diagnosis for an individual including (a) extracting cell free DNA or genomic DNA that may contain cell free tumor DNA from a liquid biopsy from the individual, wherein the cell free tumor DNA sequence has a different methylation pattern compare with normal somatic cells, (b) separating the double stranded DNA sequences into upper and lower template strands, (c) extending the upper and lower template strands using a polymerase, nucleotides and selected sets of primers which targets genomic regions that cancer cell and normal cell has different methylation patterns, resulting in hemi-methylated double stranded DNA sequences for selected differential methylated loci, (d) treating the hemi-methylated double stranded DNA sequences with methyl-transferase to add methyl groups at positions corresponding to methylated cytosine in the corresponding double stranded DNA sequences to produce fully methylated double stranded DNA sequences of selected differential methylated loci, (e) repeating steps (b) to (d) to produce fully methylated amplicons of the selected differential methylated loci, (f) treating the fully methylated amplicons with a reagent to convert cytosine residues to uracil while keeping methylated cytosine residues unchanged, (g) determining methylated cytosine pattern, (h) determine whether cell free tumor DNA exist in the sample by comparing the methylated cytosine pattern of cell free DNA with the methylated cytosine pattern of different cancer tissue, determining differences between the methylated cytosine pattern of cell free DNA and the methylated cytosine pattern of different cancer tissue, determine whether cell free tumor DNA exist in the sample; and (i) diagnosing the individual with certain type of cancer when the determined methylated cytosine pattern contains cancer specific methylation pattern. According to one aspect, the liquid biopsy sample is a blood sample, spinal fluid sample or urine sample. According to one aspect, step (c) includes magnesium ions and the treating of step (d) includes adding a chelating agent to chelate magnesium ions. According to one aspect, step (c) includes magnesium ions and the treating of step (d) includes adding EDTA to chelate magnesium ions. According to one aspect, step (c) includes magnesium ions and the treating of step (d) includes adding EDTA to chelate magnesium ions in an equal molar fashion to create an ideal buffer condition for methyl-transferase. According to one aspect, step (e) includes adding magnesium ion in repeated step (c) to create an ideal primer extension buffer condition for primer extension. According to one aspect, the methyl transferase is DNMT1. According to one aspect, the reagent to convert cytosine residues to uracil is sodium bisulfite. According to one aspect, the reagent to convert cytosine residues to uracil is an enzyme of the family APOBEC. According to one aspect, the reagent to convert cytosine residues to uracil is APOBEC3A. According to one aspect, the double stranded DNA sequence is whole genomic DNA obtained from a single cell. According to one aspect, the double stranded DNA sequence is genomic DNA from cancer cell or a circulating tumor cell. According to one aspect, the double stranded DNA sequence is cell free tumor cell genomic DNA obtained from a blood sample from an individual. According to one aspect, steps (b) to (d) are repeated between 1 to 20 times. According to one aspect, steps (b) to (d) are repeated between 1 to 10 times. According to one aspect, steps (b) to (d) are repeated between 1 to 5 times. According to one aspect, the primers are cancer specific primers. According to one aspect, determining methylated cytosine patterns includes Next-generation sequencing, methylation specific qPCR, or a methylation detecting micro-array. According to one aspect, the cancer is a member selected from the group consisting of breast invasive carcinoma, colon adenocarcinoma, liver hepatocellular carcinoma, prostate adenocarcinoma, stomach adenocarcinoma, and uterine corpus endometrial carcinoma.

What is claimed is:

1. A method of making an amplified methylome comprising
(a) fragmenting a double stranded DNA sequence having a methylation pattern to produce fragment template double stranded DNA sequences having a methylation pattern and including a primer binding site on each 5′ end and 3′ end of the fragment template double stranded DNA sequences,
(b) separating the fragment template double stranded DNA sequences into upper and lower template strands,
(c) extending the upper and lower template strands using primers, a polymerase and nucleotides to produce non-methylated complementary strands resulting in hemi-methylated double stranded DNA sequences corresponding to the fragment template double stranded DNA sequences having a methylation pattern,
(d) treating the hemi-methylated double stranded DNA sequences with methyl transferase and a source of methyl groups to add methyl groups at positions corresponding to methylated cytosine in the corresponding fragment template double stranded DNA sequences to produce fully methylated fragment template double stranded DNA sequences; and
(e) repeating steps (b) to (d) to produce fully methylated amplicons of the fragment template double stranded DNA sequences.

2. The method of claim 1 further including treating the fully methylated amplicons of the fragment template double stranded DNA sequences with a reagent to convert cytosine residues to uracil and analyzing methylated cytosine pattern.

3. The method of claim 1 wherein the fragmenting in step (a) results from contacting the double stranded DNA sequence with a library of transposomes with each transposome of the library having its own unique associated barcode sequence, wherein each transposome of the library includes a transposase and a transposon DNA homo dimer, wherein each transposon DNA of the homo dimer includes a transposase binding site, a unique barcode sequence and a primer binding site, wherein the library of transposomes bind to target locations along the double stranded DNA sequence and the transposase cleaves the double stranded DNA sequence into the fragment template double stranded DNA sequences, with each fragment template double stranded DNA sequence including one member of a unique barcode sequence pair on each end of the fragmente template double stranded DNA sequence,
gap filling a gap between the transposon DNA and the fragment template double stranded DNA sequence to form a library of fragment template double stranded DNA sequences having primer binding sites at each end.

4. The method of claim 1 wherein step (c) includes magnesium ions and the treating of step (d) includes adding a chelating agent to chelate magnesium ions.

5. The method of claim 1 wherein step (c) includes magnesium ions and the treating of step (d) includes adding EDTA to chelate magnesium ions.

6. The method of claim 1 wherein step (c) includes magnesium ions and the treating of step (d) includes adding EDTA to chelate magnesium ions in an equal molar fashion to create an ideal buffer condition for methyl-transferase.

7. The method of claim 1 wherein step (e) includes adding magnesium ion in repeated step (c) to create an ideal primer extension buffer condition for primer extension.

8. The method of claim 1 wherein the methyl transferase is DNMT1.

9. The method of claim 3 wherein the transposase is Tn5 transposase, Mu transposase, Tn7 transposase or IS5 transposase.

10. The method of claim 2 wherein the reagent to convert cytosine residues to uracil is sodium bisulfite.

11. The method of claim 1 wherein the double stranded DNA sequence is genomic DNA.

12. The method of claim 1 wherein the double stranded DNA sequence is whole genomic DNA obtained from a single cell or is cell free DNA.

13. The method of claim 1 wherein the double stranded DNA sequence is genomic DNA from a prenatal cell, a cancer cell, or a circulating tumor cell.

14. The method of claim 1 wherein the double stranded DNA sequence is cell free tumor cell genomic DNA obtained from a blood sample from an individual.

15. The method of claim 1 wherein steps (b) to (d) are repeated between 1 to 20 times.

16. The method of claim 1 wherein steps (b) to (d) are repeated between 1 to 10 times.

17. The method of claim 1 wherein steps (b) to (d) are repeated between 1 to 5 times.

18. The method of claim 1 wherein the fully methylated amplicons of the fragment template double stranded DNA sequences are treated with a reagent to convert cytosine residues to uracil.

19. The method of claim 2 wherein the reagent to convert cytosine residues to uracil is an enzyme of the family APOBEC.

20. The method of claim 2 wherein the reagent to convert cytosine residues to uracil is APOBEC3A.

21. The method of claim 1 wherein the primers are loci specific primers.

22. The method of claim 1 wherein the primers are disease specific primers.

23. The method of claim 1 wherein the primers are cancer specific primers.

24. A method of diagnosing an individual with cancer comprising
(a) fragmenting a double stranded DNA sequence obtained from a liquid biopsy sample from the individual, wherein the double stranded DNA sequence has a methylation pattern, to produce fragment template double stranded DNA sequences having a methylation pattern and including a primer binding site on each 5′ end and 3′ end of the fragment template double stranded DNA sequences,
(b) separating the fragment template double stranded DNA sequences into upper and lower template strands,
(c) extending the upper and lower template strands using cancer specific primers, a polymerase and nucleotides to produce non-methylated complementary strands resulting in hemi-methylated double stranded DNA sequences corresponding to the fragment template double stranded DNA sequences having a methylation pattern,
(d) treating the hemi-methylated double stranded DNA sequences with methyl transferase and a source of methyl groups to add methyl groups at positions corresponding to methylated cytosine in the corresponding fragment template double stranded DNA sequences to produce fully methylated fragment template double stranded DNA sequences;
(e) repeating steps (b) to (d) to produce fully methylated amplicons of the fragment template double stranded DNA sequences;
treating the fully methylated amplicons of the fragment template double stranded DNA sequences with a reagent to convert cytosine residues to uracil;
determining methylated cytosine pattern;
comparing the methylated cytosine pattern to a standard methylated cytosine pattern for cancer DNA;
determining differences between the methylated cytosine pattern and the standard methylated cytosine pattern for cancer DNA; and
diagnosing the individual with cancer when the determined methylated cytosine pattern matches the standard methylated cytosine pattern for cancer DNA.

25. The method of claim 24 wherein the liquid biopsy sample is a blood sample, spinal fluid sample or urine sample.

26. The method of claim 24 wherein step (c) includes magnesium ions and the treating of step (d) includes adding a chelating agent to chelate magnesium ions.

27. The method of claim 24 wherein step (c) includes magnesium ions and the treating of step (d) includes adding EDTA to chelate magnesium ions.

28. The method of claim 24 wherein step (c) includes magnesium ions and the treating of step (d) includes adding EDTA to chelate magnesium ions in an equal molar fashion to create an ideal buffer condition for methyl-transferase.

29. The method of claim 24 wherein step (e) includes adding magnesium ion in repeated step (c) to create an ideal primer extension buffer condition for primer extension.

30. The method of claim 24 wherein the methyl transferase is DNMT1.

31. The method of claim 24 wherein the reagent to convert cytosine residues to uracil is sodium bisulfite.

32. The method of claim 24 wherein the reagent to convert cytosine residues to uracil is an enzyme of the family APOBEC.

33. The method of claim 24 wherein the reagent to convert cytosine residues to uracil is APOBEC3A.

34. The method of claim 24 wherein the double stranded DNA sequence is whole genomic DNA obtained from a single cell.

35. The method of claim 24 wherein the double stranded DNA sequence is genomic DNA from cancer cell or a circulating tumor cell.

36. The method of claim 24 wherein the double stranded DNA sequence is cell free tumor cell genomic DNA obtained from a blood sample from an individual.

37. The method of claim 24 wherein steps (b) to (d) are repeated between 1 to 20 times.

38. The method of claim 24 wherein steps (b) to (d) are repeated between 1 to 10 times.

39. The method of claim 24 wherein steps (b) to (d) are repeated between 1 to 5 times.

40. The method of claim 24 wherein the primers are cancer specific primers.

41. The method of claim 24 wherein determining methylated cytosine patterns includes Next-generation sequencing, methylation specific qPCR, or a methylation detecting micro-array.

42. The method of claim 24 wherein the cancer is a member selected from the group consisting of breast invasive carcinoma, colon adenocarcinoma, liver hepatocellular carcinoma, prostate adenocarcinoma, stomach adenocarcinoma, and uterine corpus endometrial carcinoma.

43. A method of early cancer diagnosis for an individual comprising
(a) extracting cell free DNA or genomic DNA that may contain cell free tumor DNA from a liquid biopsy from the individual, wherein the cell free tumor DNA sequence has a different methylation pattern compare with normal somatic cells,
(b) separating the double stranded DNA sequences into upper and lower template strands,
(c) extending the upper and lower template strands using a polymerase, nucleotides and selected sets of primers which targets genomic regions that cancer cell and normal cell has different methylation patterns, resulting in hemi-methylated double stranded DNA sequences for selected differential methylated loci,
(d) treating the hemi-methylated double stranded DNA sequences with methyl-transferase to add methyl groups at positions corresponding to methylated cytosine in the corresponding double stranded DNA sequences to produce fully methylated double stranded DNA sequences of selected differential methylated loci,
(e) repeating steps (b) to (d) to produce fully methylated amplicons of the selected differential methylated loci,
(f) treating the fully methylated amplicons with a reagent to convert cytosine residues to uracil while keeping methylated cytosine residues unchanged,
(g) determining methylated cytosine pattern,
(h) determine whether cell free tumor DNA exist in the sample by comparing the methylated cytosine pattern of cell free DNA with the methylated cytosine pattern of different cancer tissue, determining differences between the methylated cytosine pattern of cell free DNA and the methylated cytosine pattern of different cancer tissue, determine whether cell free tumor DNA exist in the sample; and
(i) diagnosing the individual with certain type of cancer when the determined methylated cytosine pattern contains cancer specific methylation pattern.

44. The method of claim 43 wherein the liquid biopsy sample is a blood sample, spinal fluid sample or urine sample.

45. The method of claim 43 wherein step (c) includes magnesium ions and the treating of step (d) includes adding a chelating agent to chelate magnesium ions.

46. The method of claim 43 wherein step (c) includes magnesium ions and the treating of step (d) includes adding EDTA to chelate magnesium ions.

47. The method of claim 43 wherein step (c) includes magnesium ions and the treating of step (d) includes adding EDTA to chelate magnesium ions in an equal molar fashion to create an ideal buffer condition for methyl-transferase.

48. The method of claim 43 wherein step (e) includes adding magnesium ion in repeated step (c) to create an ideal primer extension buffer condition for primer extension.

49. The method of claim 43 wherein the methyl transferase is DNMT1.

50. The method of claim 43 wherein the reagent to convert cytosine residues to uracil is sodium bisulfite.

51. The method of claim 43 wherein the reagent to convert cytosine residues to uracil is an enzyme of the family APOBEC.

52. The method of claim 43 wherein the reagent to convert cytosine residues to uracil is APOBEC3A.

53. The method of claim 43 wherein the double stranded DNA sequence is whole genomic DNA obtained from a single cell.

54. The method of claim 43 wherein the double stranded DNA sequence is genomic DNA from cancer cell or a circulating tumor cell.

55. The method of claim 43 wherein the double stranded DNA sequence is cell free tumor cell genomic DNA obtained from a blood sample from an individual.

56. The method of claim 43 wherein steps (b) to (d) are repeated between 1 to 20 times.

57. The method of claim 43 wherein steps (b) to (d) are repeated between 1 to 10 times.

58. The method of claim 43 wherein steps (b) to (d) are repeated between 1 to 5 times.

59. The method of claim 43 wherein the primers are cancer specific primers.

60. The method of claim 43 wherein determining methylated cytosine patterns includes Next-generation sequencing, methylation specific qPCR, or a methylation detecting micro-array.

61. The method of claim 43 wherein the cancer is a member selected from the group consisting of breast invasive carcinoma, colon adenocarcinoma, liver hepatocellular carcinoma, prostate adenocarcinoma, stomach adenocarcinoma, and uterine corpus endometrial carcinoma.
